(19) United States
(12) Patent Application Publication
(10) Pub. No.: US 2007/0208165 A1
(43) Pub. Date: Sep. 6, 2007

(54) Process for the preparation of benzotriazoles

(76) Inventors: Walter Fischer, Reinach (CH); Katharina Fritzsche, Weil am Rhein (DE); Walter Wolf, Bad Sackingen (DE); Lothar Bore, Rheinfelden (DE)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/789,737

(22) Filed: Apr. 25, 2007

Related U.S. Application Data
(63) Continuation of application No. 10/380,591, filed on Mar. 17, 2003, now abandoned.

(30) Foreign Application Priority Data
Sep. 20, 2000 (CH) 1830/00

(51) Int. Cl. C07D 249/16; C07C 245/00

(52) U.S. Cl. 534/573; 548/260

(57) ABSTRACT

A process for the preparation of compounds of formula I 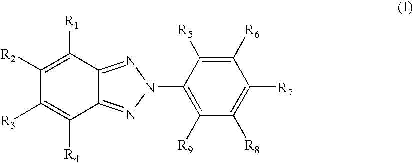
wherein the general symbols are as defined in claim 1, which comprises reacting a compound of formula V 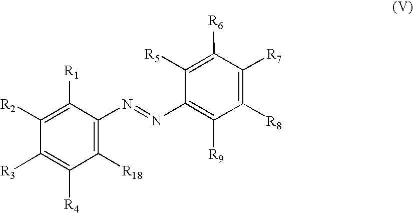
wherein R₁, R₂, R₃, R₄, R₅, R₆, R₇, R₈, R₉ and R₁₈ are as defined in claim 1, and R₁₈ is especially nitro, chlorine or bromine, with an azide compound of formula IX, 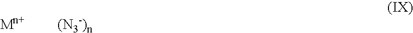
wherein M and n are as defined in claim 1, especially with sodium azide.

This application is a continuation of U.S. application Ser. No. 10/380,591, pending, which is a 371 of international app. No. PCT/EP 01/10478, filed Sep. 11, 2001, the contents of which applications are incorporated by reference.

The present invention relates to a process for the preparation of benzotriazoles, which are suitable for the stabilisation of organic materials against light-induced degradation.

The best methods used hitherto for the preparation of benzotriazoles are all based on processes in which it is necessary for a reducing agent to be used at least once. Such processes are described, for example, in U.S. Pat. No. 5,276,161 and U.S. Pat. No. 5,977,219. The reduction step produces intensely coloured, undesired secondary products, which have to be removed from the desired benzotriazoles, for example by chromatography, at great expense.

There is accordingly still a need to find an efficient process for the preparation of benzotriazoles that yields the benzotriazoles, for example, without the use of reducing agents, and accordingly does not have the above-mentioned disadvantages.

The present invention accordingly relates to a process for the preparation of compounds of formula I

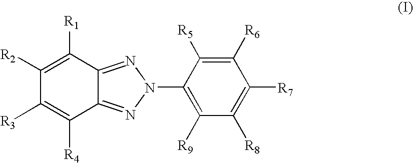

wherein

R₁, R₂, R₃, R₄, R₅, R₆, R₇, R₈ and R₉ are each independently of the others hydrogen, halogen, —SO₃H, —SO₃⁻Mₐ⁺, hydroxy, carboxy, cyano, nitro, C₁-C₂₅alkyl, C₁-C₂₅alkyl substituted by halogen, hydroxy, carboxy, cyano, C₁-C₁₈alkoxycarbonyl, C₁-C₄alkoxy or by amino; C₂-C₂₄-alkenyl, C₇-C₉phenylalkyl, unsubstituted or C₁-C₄alkyl-substituted phenyl, unsubstituted or C₁-C₄alkyl-substituted C₅-C₈cycloalkyl; C₁-C₁₈alkoxy, C₁-C₁₈alkylthio, C₁-C₁₈alkylsulfonyl, unsubstituted or C₁-C₄alkyl-substituted phenylsulfonyl, unsubstituted or C₁-C₄alkyl-substituted phenylthio; amino, C₁-C₄alkylamino, di(C₁-C₄alkyl)amino, C₁-C₂₅alkanoyl, C₁-C₂₅-alkoxycarbonyl, C₁-C₂₅alkanoyloxy, C₁-C₂₅alkanoylamino, C₃-C₂₅alkyl interrupted by oxygen, sulfur or by 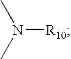
C₃-C₂₅alkoxy interrupted by oxygen, sulfur or by 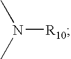
C₃-C₂₅alkanoyloxy interrupted by oxygen, sulfur or by 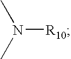
C₃-C₂₅alkoxycarbonyl interrupted by oxygen, sulfur or by 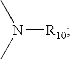
C₆-C₉cycloalkoxycarbonyl, C₆-C₉cycloalkylcarbonyloxy, unsubstituted or C₁-C₁₂alkyl-substituted benzoyloxy; —(CH₂)ₚ—COR₁₁ or —(CH₂)_{q}OH, or, further, the radicals R₆ and R₇ or the radicals R₇ and R₈ or the radicals R₈ and R₉, together with the carbon atoms to which they are bonded, form a benzo ring, R₃ in addition is a radical of formula II

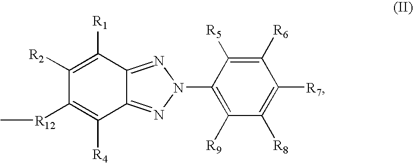

and R₆ in addition is a radical of formula III

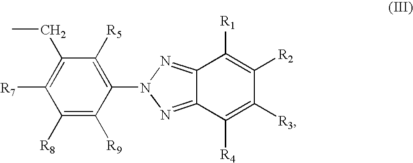

R₁₀ is hydrogen or C₁-C₈alkyl, R₁₁ is hydroxy, 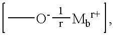
C₁-C₁₈alkoxy, 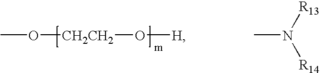
or a radical of formula IV

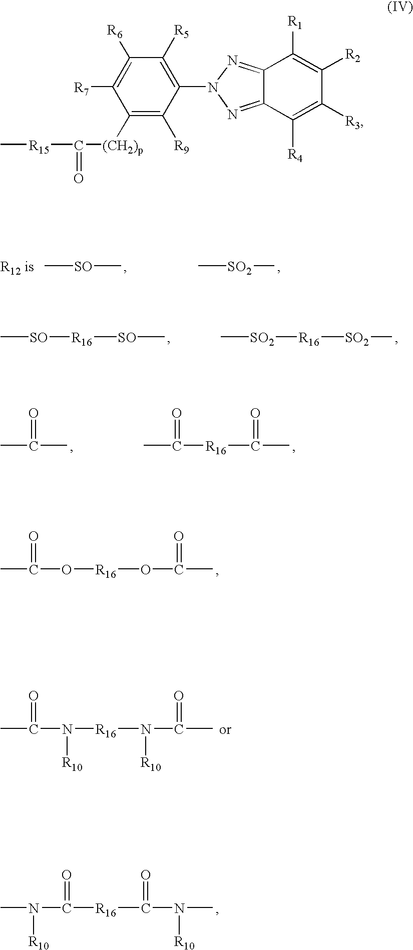

R₁₃ and R₁₄ are each independently of the other hydrogen or C₁-C₁₈alkyl,

R₁₅ is —O—R₁₇—O—,

R₁₆ is C₂-C₁₂alkylene, C₅-C₁₂cycloalkylene, or C₈-C₁₂alkylene interrupted or terminated by cyclohexylene;

R₁₇ is C₂-C₁₂alkylene, or C₄-C₁₂alkylene interrupted by oxygen, sulfur or by

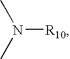

Mₐ is a monovalent metal cation,

M_{b} is an r-valent metal cation,

m is an integer from 1 to 20,

p is 0, 1 or 2,

q is 1, 2, 3, 4, 5 or 6, and

r is 1, 2 or 3,

which process comprises reacting a compound of formula V 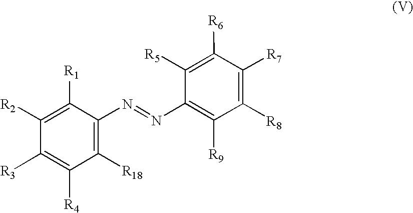
wherein

R₁, R₂, R₃, R₄, R₅, R₆, R₇, R₈ and R₉ are as defined hereinbefore, R₃ in addition being a radical of formula VI

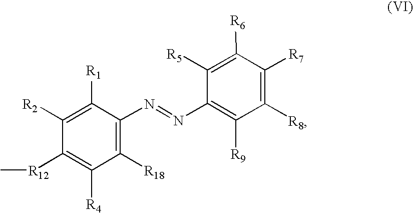

R₆ in addition being a radical of formula VII 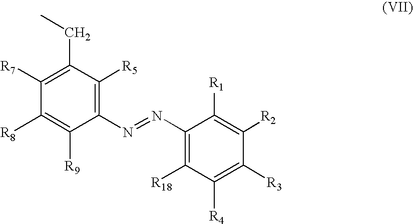
and R₁₁ in addition being a radical of formula VIII

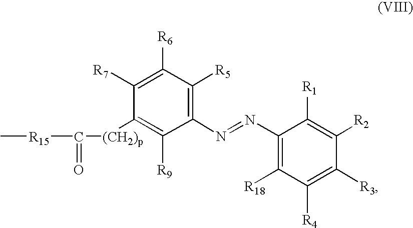

R₁₈ is halogen, nitro, 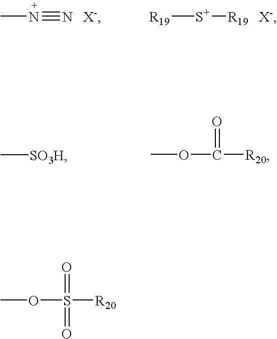
or C₁-C₁₈alkoxy,

R₁₉ is C₁-C₁₈alkyl, unsubstituted or C₁-C₄alkyl-substituted phenyl; or unsubstituted or C₁-C₄-alkyl-substituted C₅-C₈cycloalkyl,

R₂₀ is C₁-C₁₈alkyl, unsubstituted or C₁-C₄alkyl-, halo- or nitro-substituted phenyl; unsubstituted or C₁-C₄alkyl-substituted C₅-C₈cycloalkyl; or fluorine-substituted C₁-C₁₈alkyl, and

X⁻ is chloride, bromide, iodide, hydroxide, nitrate or nitrite,

with an azide compound of formula IX 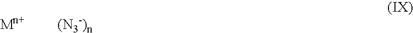
wherein

M is an n-valent metal cation,

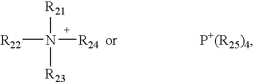

R₂₁, R₂₂, R₂₃ and R₂₄ are each independently of the others hydrogen or C₁-C₁₈alkyl,

R₂₅ is C₁-C₁₈alkyl, and

n is 1, 2 or 3.

Halogen is, for example, fluorine, chlorine, bromine or iodine. Chlorine is preferred.

Alkyl having up to 25 carbon atoms is a branched or unbranched radical, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, octadecyl, eicosyl or docosyl. One of the preferred meanings of R₁, R₂, R₃, R₄, R₅, R₇ and R₉ is, for example, C₁-C₁₈alkyl, especially C₁-C₁₂alkyl, e.g. C₁-C₄alkyl. An especially preferred meaning of R₆ is C₁-C₁₂alkyl, especially C₁-C₈alkyl, e.g. C₁-C₅alkyl. An especially preferred meaning of R₈ is C₁-C₁₂alkyl, especially C₁-C₁₀alkyl, e.g. C₁-C₈alkyl. A preferred meaning of R₁₀ is C₁-C₈alkyl, especially C₁-C₆alkyl, e.g. C₁-C₄alkyl. One of the preferred meanings of R₁₃ and R₁₄ is C₁-C₁₂alkyl, especially C₁-C₈alkyl, e.g. methyl or ethyl. A preferred meaning of R₂₀ is C₁-C₁₂alkyl, especially C₁-C₈alkyl, e.g. C₁-C₄alkyl. One of the preferred meanings of R₂₁, R₂₂, R₂₃ and R₂₄ is C₁-C₁₂alkyl, especially C₁-C₄alkyl, e.g. n-butyl.

C₁-C₂₅Alkyl substituted by halogen, hydroxy, carboxy, cyano, C₁-C₁₈alkoxycarbonyl, C₁-C₄-alkoxy or by amino is a branched or unbranched radical, for example trifluoromethyl, hydroxymethyl, carboxymethyl, cyanomethyl, methoxycarbonylmethyl, ethoxycarbonylmethyl, aminomethyl, pentafluoroethyl, 1-hydroxyethyl, 2-hydroxyethyl, 1-carboxyethyl, 2-carboxyethyl, 1-cyanoethyl, 2-cyanoethyl, 1-methoxycarbonylethyl, 2-methoxycarbonylethyl, 1-ethoxycarbonylethyl, 2-ethoxycarbonylethyl, 2-methoxyethyl, 1-aminoethyl, 2-aminoethyl, 3-chloropropyl, 1-hydroxypropyl, 2-hydroxypropyl, 3-hydroxypropyl, 1-carboxypropyl, 2-carboxypropyl, 3-carboxypropyl, 1-cyanopropyl, 2-cyanopropyl, 3-cyanopropyl, 1-methoxycarbonylpropyl, 2-methoxycarbonylpropyl, 3-methoxycarbonylpropyl, 2-methoxypropyl, 3-methoxypropyl, 1-aminopropyl, 2-aminopropyl or 3-aminopropyl.

Alkenyl having from 2 to 24 carbon atoms is a branched or unbranched radical, for example vinyl, propenyl, 2-butenyl, 3-butenyl, isobutenyl, n-2,4-pentadienyl, 3-methyl-2-butenyl, n-2-octenyl, n-2-dodecenyl, isododecenyl, oleyl, n-2-octadecenyl or n-4-octadecenyl. Preference is given to alkenyl having from 3 to 18, especially from 3 to 12, for example from 2 to 6, carbon atoms.

C₇-C₉Phenylalkyl is, for example, benzyl, α-methylbenzyl, α,α-dimethylbenzyl or 2-phenylethyl. A preferred meaning of R₆ and R₈ is, for example, α,α-dimethylbenzyl.

Unsubstituted or C₁-C₄alkyl-, halo- or nitro-substituted phenyl that contains preferably from 1 to 3 substituents, especially 1 or 2 substituents, is, for example, o-, m- or p-methylphenyl, p-chlorophenyl, p-nitrophenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2-methyl-6-ethylphenyl, 4-tertbutylphenyl, 2-ethylphenyl, 2,6-diethylphenyl, 2,4-dichlorophenyl, 2,4,6-trichlorophenyl, 2,4-dinitrophenyl or 2,6-dichloro-4-nitrophenyl.

Unsubstituted or C₁-C₄alkyl-substituted C₅-C₈cycloalkyl is, for example, cyclopentyl, methylcyclopentyl, dimethylcyclopentyl, cyclohexyl, methylcyclohexyl, dimethylcyclohexyl, trimethylcyclohexyl, tert-butylcyclohexyl, cycloheptyl or cyclooctyl. Preference is given to cyclohexyl.

Alkoxy having up to 18 carbon atoms is a branched or unbranched radical, for example methoxy, ethoxy, propoxy, isopropoxy, n-butoxy, isobutoxy, pentyloxy, isopentyloxy, hexyloxy, heptyloxy, octyloxy, decyloxy, tetradecyloxy, hexadecyloxy or octadecyloxy. Preference is given to alkoxy having from 1 to 12, especially from 1 to 8, e.g. from 1 to 6, carbon atoms. An especially preferred meaning of R₂, R₁₁ and R₁₈ is methoxy.

Alkylthio having up to 25 carbon atoms is a branched or unbranched radical, for example methylthio, ethylthio, propylthio, isopropylthio, n-butylthio, isobutylthio, pentylthio, isopentylthio, hexylthio, heptylthio, octylthio, decylthio, tetradecylthio, hexadecylthio or octadecylthio. Preference is given to alkylthio having from 1 to 12, especially from 1 to 8, e.g. from 1 to 6, carbon atoms.

Alkylsulfonyl having up to 18 carbon atoms is a branched or unbranched radical, for example methylsulfonyl, ethylsulfonyl, n-propylsulfonyl, isopropylsulfonyl, n-butylsulfonyl, isobutylsulfonyl, tert-butylsulfonyl, 2-ethylbutylsulfonyl, n-pentylsulfonyl, isopentylsulfonyl, 1-methylpentylsulfonyl, 1,3-dimethylbutylsulfonyl, n-hexylsulfonyl, 1-methylhexylsulfonyl, n-heptylsulfonyl, isoheptylsulfonyl, 1,1,3,3-tetramethylbutylsulfonyl, 1-methylheptylsulfonyl, 3-methylheptylsulfonyl, n-octylsulfonyl, 2-ethylhexylsulfonyl, 1,1,3-trimethylhexylsulfonyl, 1,1,3,3-tetramethylpentylsulfonyl, nonylsulfonyl, decylsulfonyl, undecylsulfonyl, 1-methylundecylsulfonyl, dodecylsulfonyl, 1,1,3,3,5,5-hexamethylhexylsulfonyl, tridecylsulfonyl, tetradecylsulfonyl, pentadecylsulfonyl, hexadecylsulfonyl, heptadecylsulfonyl or octadecylsulfonyl. A preferred meaning of R₂ and R₃ is C₁-C₁₂alkylsulfonyl, especially C₁-C₈alkylsulfonyl, e.g. C₁-C₄alkylsulfonyl.

Unsubstituted or C₁-C₄alkyl-substituted phenylsulfonyl that contains preferably from 1 to 3 alkyl groups, especially 1 or 2 alkyl groups, is, for example, o-, m- or p-methylphenylsulfonyl, 2,3-dimethylphenylsulfonyl, 2,4-dimethylphenylsulfonyl, 2,5-dimethylphenylsulfonyl, 2,6-dimethylphenylsulfonyl, 3,4-dimethylphenylsulfonyl, 3,5-dimethylphenylsulfonyl, 2-methyl-6-ethylphenylsulfonyl, 4-tert-butylphenylsulfonyl, 2-ethylphenylsulfonyl or 2,6-diethylphenylsulfonyl.

Unsubstituted or C₁-C₄alkyl-substituted phenylthio that contains preferably from 1 to 3 alkyl groups, especially 1 or 2 alkyl groups, is, for example, o-, m- or p-methylphenylthio, 2,3-dimethylphenylthio, 2,4-dimethylphenylthio, 2,5-dimethylphenylthio, 2,6-dimethylphenylthio, 3,4-dimethylphenylthio, 3,5-dimethylphenylthio, 2-methyl-6-ethylphenylthio, 4-tert-butylphenylthio, 2-ethylphenylthio or 2,6-diethylphenylthio.

Alkylamino having up to 4 carbon atoms is a branched or unbranched radical, for example methylamino, ethylamino, propylamino, isopropylamino, n-butylamino, isobutylamino or tertbutylamino.

Di(C₁-C₄alkyl)amino denotes that the two radicals are each independently of the other branched or unbranched, for example dimethylamino, methylethylamino, diethylamino, methyl-n-propylamino, methylisopropylamino, methyl-n-butylamino, methylisobutylamino, ethylisopropylamino, ethyl-n-butylamino, ethylisobutylamino, ethyl-tert-butylamino, diethylamino, diisopropylamino, isopropyl-n-butylamino, isopropylisobutylamino, di-n-butylamino or di-isobutylamino.

Alkanoyl having up to 25 carbon atoms is a branched or unbranched radical, for example formyl, acetyl, propionyl, butanoyl, pentanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, eicosanoyl or docosanoyl. Alkanoyl has preferably from 2 to 18, especially from 2 to 12, e.g. from 2 to 6, carbon atoms. Special preference is given to acetyl.

Alkoxycarbonyl having up to 25 carbon atoms is a branched or unbranched radical, for example methoxycarbonyl, ethoxycarbonyl, propoxycarbonyl, isopropoxycarbonyl, n-butoxycarbonyl, isobutoxycarbonyl, pentyloxycarbonyl, isopentyloxycarbonyl, hexyloxycarbonyl, heptyloxycarbonyl, octyloxycarbonyl, decyloxycarbonyl, tetradecyloxycarbonyl, hexadecyloxycarbonyl or octadecyloxycarbonyl. Preference is given to alkoxycarbonyl having from 1 to 18, especially from 1 to 12, e.g. from 1 to 8, carbon atoms. An especially preferred meaning is methoxycarbonyl or ethoxycarbonyl.

Alkanoyloxy having up to 25 carbon atoms is a branched or unbranched radical, for example formyloxy, acetoxy, propionyloxy, butanoyloxy, pentanoyloxy, hexanoyloxy, heptanoyloxy, octanoyloxy, nonanoyloxy, decanoyloxy, undecanoyloxy, dodecanoyloxy, tridecanoyloxy, tetradecanoyloxy, pentadecanoyloxy, hexadecanoyloxy, heptadecanoyloxy, octadecanoyloxy, eicosanoyloxy or docosanoyloxy. Preference is given to alkanoyloxy having from 2 to 18, especially from 2 to 12, e.g. from 2 to 6, carbon atoms. Special preference is given to acetoxy.

Alkanoylamino having up to 25 carbon atoms is a branched or unbranched radical, for example formylamino, acetylamino, propionylamino, butanoylamino, pentanoylamino, hexanoylamino, heptanoylamino, octanoylamino, nonanoylamino, decanoylamino, undecanoylamino, dodecanoylamino, tridecanoylamino, tetradecanoylamino, pentadecanoylamino, hexadecanoylamino, heptadecanoylamino, octadecanoylamino, eicosanoylamino or docosanoylamino. Preference is given to alkanoylamino having from 2 to 18, especially from 2 to 12, e.g. from 2 to 6, carbon atoms.

C₃-C₂₅Alkyl interrupted by oxygen, sulfur or by 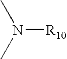
is, for example, CH₃—O—CH₂CH₂—,

CH₃—S—CH₂CH₂—, CH₃—N(CH₃)—CH₂—, CH₃—O—CH₂CH₂—O—CH₂CH₂—,

CH₃—(O—CH₂CH₂—)₂O—CH₂CH₂—, CH₃—(O—CH₂CH₂—)₃O—CH₂CH₂— or

CH₃—(O—CH₂CH₂—)₄O—CH₂CH₂—.

C₃-C₂₅Alkoxy interrupted by oxygen, sulfur or by 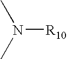
is, for example,

CH₃—O—CH₂CH₂O—, CH₃—S—CH₂CH₂O—, CH₃—N(CH₃)—CH₂CH₂O—,

CH₃—O—CH₂CH₂—O—CH₂CH₂O—, CH₃—(O—CH₂CH₂—)₂O—CH₂CH₂O—,

CH₃—(O—CH₂CH₂—)₃O—CH₂CH₂O— or CH₃—(O—CH₂CH₂—)₄O—CH₂CH₂O—.

C₃-C₂₅Alkanoyloxy interrupted by oxygen, sulfur or by 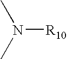
is, for example,

CH₃—O—CH₂COO—, CH₃—S—CH₂COO—, CH₃—N(CH₃)—CH₂COO—,

CH₃—O—CH₂CH₂—O—CH₂COO—, CH₃—(O—CH₂CH₂—)₂O—CH₂COO—,

CH₃—(O—CH₂CH₂—)₃O—CH₂COO— or CH₃—(O—CH₂CH₂—)₄O—CH₂COO—.

C₃-C₂₅Alkoxycarbonyl interrupted by oxygen, sulfur or by 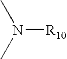
is, for example,

CH₃—O—CH₂CH₂OCO—, CH₃—S—CH₂CH₂OCO—, CH₃—N(CH₃)—CH₂CH₂OCO—,

CH₃—O—CH₂CH₂—O—CH₂CH₂OCO—, CH₃—(O—CH₂CH₂—)₂O—CH₂CH₂OCO—,

CH₃—(O—CH₂CH₂—)₃O—CH₂CH₂OCO— or CH₃—(O—CH₂CH₂—)₄O—CH₂CH₂OCO—.

C₆-C₉Cycloalkoxycarbonyl is, for example, cyclohexyloxycarbonyl, cycloheptyloxycarbonyl, cyclooctyloxycarbonyl or cyclononyloxycarbonyl. Preference is given to cyclohexyloxycarbonyl.

C₆-C₉Cycloalkylcarbonyloxy is, for example, cyclohexylcarbonyloxy, cycloheptylcarbonyloxy, cyclooctylcarbonyloxy or cyclononylcarbonyloxy. Preference is given to cyclohexylcarbonyloxy.

C₁-C₁₂Alkyl-substituted benzoyloxy, which carries preferably from 1 to 3 alkyl groups, especially 1 or 2 alkyl groups, is, for example, o-, m- or p-methylbenzoyloxy, 2,3-dimethylbenzoyloxy, 2,4-dimethylbenzoyloxy, 2,5-dimethylbenzoyloxy, 2,6-dimethylbenzoyloxy, 3,4-dimethylbenzoyloxy, 3,5-dimethylbenzoyloxy, 2-methyl-6-ethylbenzoyloxy, 4-tert-butylbenzoyloxy, 2ethylbenzoyloxy, 2,4,6-trimethylbenzoyloxy, 2,6-dimethyl-4-tert-butylbenzoyloxy or 3,5-di-tertbutylbenzoyloxy. Preferred substituents are C₁-C₈alkyl, especially C₁-C₄alkyl.

C₂-C₁₈Alkylene is a branched or unbranched radical, for example ethylene, propylene, trimethylene, tetramethylene, pentamethylene, hexamethylene, heptamethylene, octamethyllene, decamethylene, dodecamethylene or octadecamethylene. Preference is given to C₁-C₁₂alkylene, especially C₁-C₈alkylene.

C₅-C₁₂Cycloalkylene is a saturated hydrocarbon group having two free valences and at least one ring unit and is, for example, cyclopentylene, cyclohexylene, cycloheptylene, cyclooctylene, cyclononylene, cyclodecylene, cycloundecylene or cyclododecylene. Preference is given to cyclohexylene.

C₈-C₁₂Alkylene interrupted or terminated by cyclohexylene is, for example,

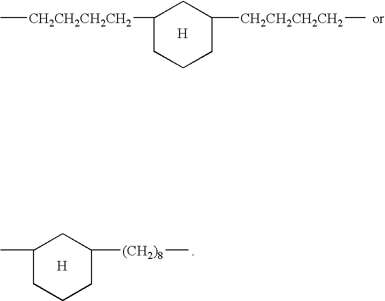

C₄-C₁₂Alkylene interrupted by oxygen, sulfur or by 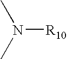
is, for example,

—CH₂CH₂—O—CH₂CH₂—, —CH₂CH₂—S—CH₂CH₂—, —CH₂CH₂—N(CH₃)—CH₂CH₂—,

—CH₂CH₂—O—CH₂CH₂—O—CH₂CH₂—, —CH₂CH₂—(O—CH₂CH₂—)₂O—CH₂CH₂—,

—CH₂CH₂—(O—CH₂CH₂—)₃O—CH₂CH₂—, —CH₂CH₂—(O—CH₂CH₂—)₄O—CH₂CH₂— or

—CH₂CH₂—S—CH₂CH₂—.

A mono-, di- or tri-valent metal cation is preferably an alkali metal, alkaline earth metal or aluminium cation, for example, Li⁺, Na⁺, K⁺, Mg⁺⁺, Ca⁺⁺ or Al⁺⁺⁺.

Fluorine-substituted C₁-C₁₈alkyl is a branched or unbranched radical, for example trifluoromethyl, pentafluoroethyl or hexafluoroisopropyl. A preferred meaning of R₂₀ is trifluoromethyl.

Preference is given to a process for the preparation of compounds of formula I wherein R₁₁ is hydroxy, 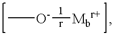
C₁-C₁₈alkoxy, 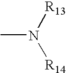
or a radical of formula IV;

R₁₃ and R₁₄ are each independently of the other hydrogen or C₁-C₁₈alkyl,

M_{b} is an r-valent metal cation, and

r is 1, 2 or 3.

Of interest is a process for the preparation of compounds of formula I wherein R₁, R₂, R₃, R₄, R₅, R₆, R₇, R₈ and R₉ are each independently of the others hydrogen, halogen, —SO₃H, —SO₃⁻Mₐ⁺, hydroxy, carboxy, cyano, nitro, C₁-C₁₈alkyl, fluorine- or C₁-C₄alkoxy-substituted C₁-C₁₈alkyl; C₂-C₁₈alkenyl, C₇-C₉phenylalkyl, unsubstituted or C₁-C₄alkyl-substituted phenyl; C₅-C₈cycloalkyl, C₁-C₁₈alkoxy, C₁-C₁₈alkylthio, C₁-C₁₈alkylsulfonyl, phenylsulfonyl, phenylthio, C₁-C₄alkylamino, di(C₁-C₄alkyl)amino, C₁-C₁₈alkanoyl, C₁-C₁₈alkoxycarbonyl, C₁-C₁₈alkanoyloxy, C₁-C₁₈alkanoylamino, C₃-C₁₈alkyl interrupted by oxygen, sulfur or by 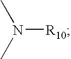
C₃-C₁₈alkoxy interrupted by oxygen, sulfur or by 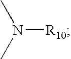
C₃-C₁₈alkanoyloxy interrupted by oxygen, sulfur or by 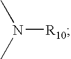
C₃-C₁₈alkoxycarbonyl interrupted by oxygen, sulfur or by 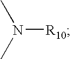
C₆-C₉cycloalkoxycarbonyl, C₆-C₉cycloalkylcarbonyloxy, unsubstituted or C₁-C₄alkyl-substituted benzoyloxy; —(CH₂)ₚ—COR₁₁ or —(CH₂)_{q}OH; or, further, the radicals R₆ and R₇ or the radicals R₇ and R₈ or the radicals R₈ and R₉, together with the carbon atoms to which they are bonded, form a benzo ring, R₃ in addition is a radical of formula II and R₆ in addition is a radical of formula III,

R₁₀ is hydrogen or C₁-C₆alkyl,

R₁₁ is hydroxy, 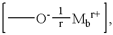
C₁-C₁₂alkoxy, 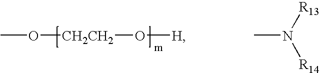
or a radical of formula IV,

R₁₂ is —SO₂—, —SO₂—R₁₆—SO₂—,

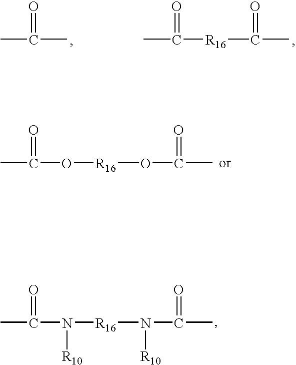

R₁₃ and R₁₄ are each independently of the other hydrogen or C₁-C₈alkyl,

R₁₅ is —O—R₁₇—O—,

R₁₆ is C₂-C₁₂alkylene or C₅-C₁₂cycloalkylene,

R₁₇ is C₂-C₈alkylene, or C₄-C₁₂alkylene interrupted by oxygen or by sulfur,

R₁₈ is halogen, nitro, 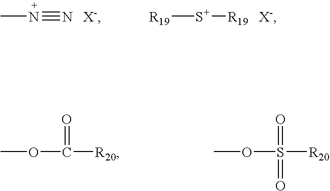
or C₁-C₁₂alkoxy,

R₁₉ is C₁-C₁₈alkyl, phenyl or C₅-C₈cycloalkyl,

R₂₀ is C₁-C₁₈alkyl, unsubstituted or C₁-C₄alkyl-, fluorine-, chlorine- or nitro-substituted phenyl;

C₅-C₈cycloalkyl, or fluorine-substituted C₁-C₁₂alkyl,

R₂₁, R₂₂, R₂₃ and R₂₄ are each independently of the others hydrogen or C₁-C₁₂alkyl,

R₂₅ is C₁-C₁₂alkyl,

M is lithium, sodium, potassium, calcium,

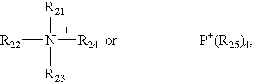

Mₐ is sodium or potassium,

M_{b} is sodium, potassium or calcium,

X⁻ is chloride or bromide,

m is an integer from 1 to 15,

n is 1 or 2,

p is 0, 1 or 2,

q is 1, 2 or 3, and

r is 1 or 2.

Also of interest is a process for the preparation of compounds of formula I wherein R₁ is hydrogen, chlorine, carboxy, nitro, C₁-C₄alkyl, trifluoromethyl or C₁-C₄alkoxy, R₂ is hydrogen, chlorine, —SO₃H, —SO₃⁻Mₐ⁺, carboxy, cyano, nitro, C₁-C₄alkyl, trifluoromethyl, C₁-C₄alkoxy, benzyl, phenyl, cyclohexyl, C₁-C₄alkylsulfonyl, phenylsulfonyl, C₁-C₈alkanoyl, C₁-C₈alkoxycarbonyl, C₁-C₈alkanoyloxy, C₃-C₈alkyl interrupted by oxygen or by sulfur; C₃-C₈-alkoxy interrupted by oxygen or by sulfur; C₃-C₈alkanoyloxy interrupted by oxygen or by sulfur; C₃-C₈alkoxycarbonyl interrupted by oxygen or by sulfur; cyclohexyloxycarbonyl, cyclohexylcarbonyloxy, or benzoyloxy,

R₃ is hydrogen or a radical of formula II,

R₄ is hydrogen, chlorine, carboxy, nitro, C₁-C₄alkyl, trifluoromethyl or C₁-C₄alkoxy,

R₅ is hydrogen, chlorine, hydroxy, C₁-C₄alkyl, C₁-C₄alkoxy, C₁-C₈alkanoyloxy, C₃-C₈alkanoyloxy interrupted by oxygen or by sulfur; C₆-C₉cycloalkylcarbonyloxy, or unsubstituted or C₁-C₄alkyl-substituted benzoyloxy,

R₆ is hydrogen, C₁-C₁₂alkyl, C₇-C₉phenylalkyl, phenyl, cyclohexyl, C₁-C₁₂alkoxy, C₃-C₁₂alkyl interrupted by oxygen or by sulfur; C₃-C₁₂alkoxy interrupted by oxygen or by sulfur; or a radical of formula III,

R₇ is hydrogen, chlorine, C₁-C₄alkyl or C₁-C₄alkoxy,

R₈ is hydrogen, C₁-C₁₂alkyl, C₇-C₉phenylalkyl, phenyl, cyclohexyl, C₁-C₁₂alkoxy, C₃-C₁₂alkyl interrupted by oxygen or by sulfur; C₃-C₁₂alkoxy interrupted by oxygen or by sulfur; or —(CH₂)ₚ—COR₁₁,

R₉ is hydrogen, chlorine, C₁-C₄alkyl or C₁-C₄alkoxy,

R₁₁ is hydroxy, C₁-C₈alkoxy, 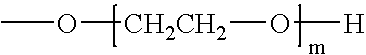
or a radical of formula IV,

R₁₂ is —SO₂—,

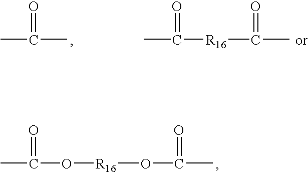

R₁₅ is —O—R₁₇—O—.

R₁₆ is C₂-C₁₈alkylene or C₅-C₈cycloalkylene,

R₁₇ is C₂-C₈alkylene, or C₄-C₁₂alkylene interrupted by oxygen,

R₁₈ is chlorine, bromine, iodine, nitro,

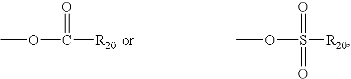

R₂₀ is C₁-C₈alkyl, unsubstituted or fluorine-, chlorine- or nitro-substituted phenyl; or fluorine-substituted C₁-C₄alkyl,

M is lithium, sodium, potassium or calcium, Mₐ⁺ is sodium or potassium,

m is an integer from 1 to 15,

n is 1 or 2, and

p is 1 or 2.

Of special interest is a process for the preparation of compounds of formula I wherein R₁, R₄, R₇ and R₉ are hydrogen.

Likewise of special interest is a process for the preparation of compounds of formula I wherein R₁₈ is nitro, chlorine or bromine.

Of particular special interest is a process for the preparation of compounds of formula I wherein

R₁ is hydrogen or C₁-C₄alkyl,

R₂ is hydrogen, chlorine, —SO₃H, —SO₃⁻ Mₐ⁺, carboxy, cyano, nitro, C₁-C₄alkyl, trifluoromethyl,

C₁-C₄alkoxy, C₁-C₄alkanoyl, C₁-C₄alkoxycarbonyl, C₃-C₈alkyl interrupted by oxygen; or C₃-C₈-alkoxy interrupted by oxygen,

R₃ is hydrogen,

R₄ is hydrogen or C₁-C₄alkyl,

R₅ is hydrogen, hydroxy, C₁-C₄alkyl, C₁-C₄alkoxy, C₁-C₈alkanoyloxy or benzoyloxy,

R₆ is hydrogen, C₁-C₈alkyl, C₇-C₉phenylalkyl, phenyl, cyclohexyl or a radical of formula III,

R₇ is hydrogen or C₁-C₄alkyl,

R₈ is hydrogen, C₁-C₁₂alkyl, C₇-C₉phenylalkyl, phenyl, cyclohexyl or —(CH₂)ₚCOR₁₁,

R₉ is hydrogen or C₁-C₄alkyl,

R₁₁ is hydroxy, C₁-C₈alkoxy, 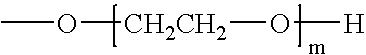
or a radical of formula IV,

R₁₅ is —O—R₁₇—O—,

R₁₇ is C₄-C₁₂alkylene interrupted by oxygen,

R₁₈ is chlorine, bromine, nitro,

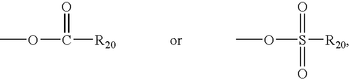

R₂₀ is C₁-C₄alkyl, unsubstituted or fluorine-, chlorine- or nitro-substituted phenyl; or trifluoromethyl,

M is lithium, sodium or potassium,

Mₐ⁺ is sodium or potassium,

m is an integer from 1 to 10,

n is 1, and

p is 2.

Special preference is given to a process for the preparation of compounds of formula I wherein

R₁ is hydrogen,

R₂ is hydrogen, chlorine, —SO₃H, —SO₃⁻Mₐ⁺, carboxy, cyano, nitro or trifluoromethyl,

R₃ is hydrogen,

R₄ is hydrogen,

R₅ is hydrogen or hydroxy,

R₆ is hydrogen, C₁-C₅alkyl, α,α-dimethylbenzyl or a radical of formula III,

R₇ is hydrogen,

R₈ is hydrogen, C₁-C₈alkyl or —(CH₂)ₚ—COR₁₁,

R₉ is hydrogen,

R₁₁ is C₁-C₈alkoxy, 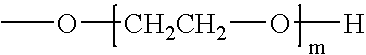
or a radical of formula IV,

R₁₅ is —O—R₁₇—O—,

R₁₇ is C₄-C₁₂alkylene interrupted by oxygen,

R₁₈ is nitro, chlorine or bromine,

M is lithium or sodium,

Mₐ⁺ is sodium or potassium,

m is an integer from 1 to 10,

n is 1, and

p is 2.

Preferred reaction conditions of the process according to the invention are as follows:

The reaction can be carried out in the melt or in a solvent. Of special interest is a process for the preparation of compounds of formula I wherein the reaction is carried out in a solvent.

Suitable solvents are, for example, dipolar aprotic solvents, protic solvents, esters of aliphatic or aromatic carboxylic acids, ethers, halogenated hydrocarbons, aromatic solvents, amines and alkoxybenzenes.

Examples of dipolar aprotic solvents are dialkyl sulfoxides, for example dimethyl sulfoxide; carboxamides, for example formamide, dimethylformamide or N,N-dimethylacetamide; lactams, for example N-methylpyrrolidone; phosphoric amides, for example hexamethylphosphoric triamide; alkylated ureas, for example N,N′-dimethylethyleneurea, N,N′-dimethylpropyleneurea or N,N,N′,N′-tetramethylurea; and nitriles, for example acetonitrile or benzonitrile.

Examples of protic solvents are polyalkylene glycols, for example polyethylene glycol; polyalkylene glycol monoethers, for example diethylene glycol monomethyl ether, and water, the latter on its own or in a single-phase or two-phase mixture with one or more of the solvents mentioned, it being possible also for phase transfer catalysts to be added, for example tetra-alkylammonium salts, tetraalkylphosphonium salts or crown ethers. The same phase transfer catalysts can also be of use in solid/liquid form in the two-phase system.

Preferred esters of aliphatic or aromatic carboxylic acids are, for example, butyl acetate, cyclohexyl acetate and methyl benzoate.

Preferred ethers are, for example, dialkyl ethers, especially dibutyl ether, tetrahydrofuran, dioxane and (poly-)alkylene glycol dialkyl ethers.

Halogenated hydrocarbons are, for example, methylene chloride and chloroform.

Aromatic solvents are, for example, toluene, chlorobenzene and nitrobenzene.

Suitable amine solvents are, for example, triethylamine, tributylamine and benzyl-dimethylamine.

Preferred alkoxybenzenes are, for example, anisole and phenetole.

The process for the preparation of compounds of formula I can also be carried out in ionic or supercritical fluids, for example fluid carbon dioxide.

Of special interest is a process for the preparation of compounds of formula I wherein the reaction is carried out in a diplar aprotic solvent.

The reaction temperatures can be varied within wide limits but are so selected that satisfactory conversion occurs, such temperatures preferably being from 10° to 180° C., especially from 20° to 150° C. The reaction is preferably so carried out that the intermediate of formula X 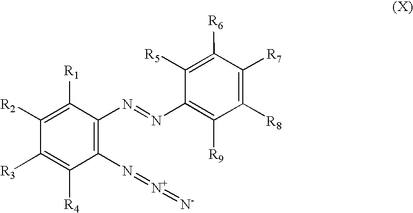
is not formed at all, or at most is formed only in a small amount and immediately reacts further to form the compound of formula I.

Preference is given to a process for the preparation of compounds of formula I wherein the molar ratio of the amount of compound of formula V to the amount of azide compound of formula IX is from 1:1 to 1:3, especially from 1:1 to 1:2, e.g. from 1:1 to 1:1.3. When functional side groups that are also able to react with azide are present, the excess of the azide compound of formula IX is increased accordingly.

When R₁₈ is a halogen atom, for example, chlorine, bromine or iodine, the reaction can be accelerated by the addition of a suitable catalyst. Such catalysts include, for example, copper(I) or copper(II) salts or other transition metal salts, based, for example, on iron, cobalt, nickel, palladium, platinum, gold or zinc. Instead of transition metal salts, the anions of which can be varied within wide limits, it is also possible to use metal complexes and metal complex salts of the same metals as catalysts. Preference is given to the use of copper(I) and copper(II) chlorides, bromides and iodides, and special preference to the use of copper(I) bromide.

Accordingly, there is also of special interest a process for the preparation of compounds of formula I wherein the reaction is carried out in the presence of a catalyst.

The catalyst is advantageously used in an amount of from 0.01 to 10% by weight, especially from 0.1 to 5% by weight, e.g. from 0.1 to 5% by weight, based on the weight of the compound of formula V employed.

The reaction can also be carried out in the presence of an additional base or in the presence of an alkaline pH buffer system. Suitable pH buffer systems include, for example, alkali metal or alkaline earth metal hydroxides; alkali metal or alkaline earth metal alcoholates; alkali metal or alkaline earth metal carboxylates, for example acetates or carbonates; alkali metal or alkaline earth metal phosphates; tertiary amines, for example triethylamine or tributylamine; and unsubstituted or substituted pyridines.

The starting materials of formula V can be used in the form of pure substances or in the form of crude solutions that comprise the compounds of formula V. The compounds of formula I can also be prepared in a so-called one-pot process. In that process the compounds of formula V are prepared in situ and are reacted with a compound of formula IX, without being isolated, to form the compounds of formula I.

Working up of the reaction mixture is advantageously carried out by evaporating off the solvent at normal pressure or in vacuo. The reaction mixture can also be diluted with water, extracted with an organic solvent, for example toluene, and then concentrated by evaporation. The residue comprising the compounds of formula I is purified by customary known methods, for example recrystallisation, precipitation, crystallisation, distillation at normal pressure or in vacuo, chromatography on silica gel or aluminium oxide, or adsorption of the polar impurities on solid phases, for example fuller's earths, activated carbon or Hyflo. The choice of working-up method depends on the physical properties of the compound of formula I and of any secondary products.

Most of the starting compounds of formula V are known from the literature or can be prepared analogously to the procedures described in Examples 9a, 10a, 12a and 13a.

The compounds of formula I are UV absorbers and are suitable as stabilisers for organic materials. Many of them are obtainable commercially from Ciba Spezialitatenchemie AG, for example Tinuvin 327 (RTM) (compound 109, Table 1); Tinuvin 328 (RTM) (compound 106, Table 1); Tinuvin 343 (RTM) (compound 103, Table 1); and Tinuvin P (RTM) (compound 104, Table 1).

The following Examples illustrate the invention further. Parts or percentages relate to weight.

### EXAMPLE 1

### Preparation of 2-phenyl-4,5-benzo-1,2,3-triazole (compound 101, Table 1)

1.0 g (4.40 mmol) of 2-nitro-azobenzene (compound 201, Table 2) and 0.34 g (5.28 mmol) of sodium azide are stirred for 10 hours at 125° C. in 5 ml of dimethyl sulfoxide. After the conversion has taken place, the mixture is cooled and toluene and water are added. The organic phase is washed repeatedly with water and then with 2N hydrochloric acid solution, dried over sodium sulfate and concentrated by evaporation in vacuo. 0.82 g (95 %) of 2-phenyl-4,5-benzo-1,2,3-triazole (compound 101, Table 1), m.p. 106-107° C. (Lit. 108-109° C., P. Spagnolo et al., J. Chem. Soc., Perkin Trans. I 1988, 2615) is obtained.

### EXAMPLE 2

### Preparation of Compound 102 (Table 1)

0.40 g (1.53 mmol) of 1-chloro-4-nitro-2-(phenylazo)-benzene (compound 202, Table 2) is dissolved in 2 g of N-methylpyrrolidone, and 0.12 g (1.83 mmol) of sodium azide is added. The mixture is stirred for 4 hours at 25° C. After the conversion has taken place, the mixture is cooled and toluene and water are added. The organic phase is washed repeatedly with water and then with 2N hydrochloric acid solution, dried over sodium sulfate and concentrated using a vacuum rotary evaporator. 0.31 g (86%) of compound 102 (Table 1), m.p. 170-172° C. (Lit. 175-177° C., P. G. Houghton et al., J. Chem. Soc., Perkin Trans I 1985, 1471) is obtained.

### EXAMPLE 3

### Preparation of Compound 103 (Table 1) Starting From Compound 203 (Table 2)

1.0 g (2.81 mmol) of 2-(2-butyl)-4-tert-butyl-6-(2-nitrophenylazo)-phenol (compound 203, Table 2) is stirred for 4 hours at 125° C. with 5 ml of dimethylformamide and 0.24 g (3.69 mmol) of sodium azide. After the reaction has taken place, the mixture is taken up in water and extracted with THF/toluene (1:1). The organic phases are washed three times with water and once with saturated sodium chloride solution, dried over sodium sulfate and concentrated using a vacuum rotary evaporator. 0.88 g (92%) of 2-(2-butyl)-4-tert-butyl-6-(4,5-benzo-1,2,3-triazol-2-yl)-phenol (compound 103, Table 1), m.p. 81-84° C., is obtained.

The use of dimethyl sulfoxide, N,N-dimethylacetamide or N-methylpyrrolidone instead of dimethylformamide under otherwise identical conditions produces very similar results.

When the reaction is carried out in dimethylformamide under otherwise identical conditions with the addition of 0.52 g (2.81 mmol) of tributylamine, the reaction is complete after 4 hours at 160° C. and the yield of compound 103 (Table 1) is 0.85 9 (89%).

When the reaction is carried out in dimethylformamide under otherwise identical conditions with the addition of 2.03 g (6.75 mmol) of polyethylene glycol 300, the reaction is complete after 4 hours at 160° C. and the yield of compound 103 (Table 1) is 0.83 9 (87%).

### EXAMPLE 4

### Preparation of Compound 103 (Table 1) Starting From Compound 204 (Table 2)

1.0 g (2.90 mmol) of 2-(2-butyl)-4-tert-butyl-6-(2-chloro-phenylazo)-phenol (compound 204, Table 2), 5 g of N,N-dimethylformamide, 0.226 g (3.48 mmol) of sodium azide and 4.2 mg (0.029 mmol; 1 mol %) of copper(I) bromide are combined and stirred for 4 hours at 80° C. After the conversion has taken place, the mixture is cooled and toluene and water are added. The organic phase is washed repeatedly with water and then with 2N hydrochloric acid solution, dried over sodium sulfate, filtered over silica gel and concentrated using a vacuum rotary evaporator. 0.90 g (92%) of 2-(2-butyl)-4-tert-butyl-6-(4,5-benzo-1,2,3-triazol-2-yl)-phenol (compound 103, Table 1), m.p. 81-84° C., is obtained.

The use of ethylene glycol monobutyl ether instead of dimethylformamide under similar conditions produces comparable results, complete conversion being achieved after 4 hours at 120° C.

The use of butyl acetate instead of dimethylformamide under similar conditions produces comparable results, almost complete conversion being achieved after stirring for one day at reflux at approximately 125° C.

### EXAMPLE 5

### Preparation of Compound 103 (Table 1) Starting From Compound 205 (Table 2)

1.0 g (2.57 mmol) of 2-(2-butyl)-4-tert-butyl-6-(2-bromo-phenylazo)-phenol (compound 205, Table 2), 5 g of N,N-dimethylformamide, 0.2 g (3.08 mmol) of sodium azide and 3.7 mg (0.0257 mmol; 1 mol %) of copper(I) bromide are combined and stirred for 2 hours at 80° C. After the conversion has taken place, the mixture is cooled and toluene and water are added. The organic phase is washed repeatedly with water and then with 2N hydrochloric acid solution, dried over sodium sulfate, filtered over silica gel and concentrated using a vacuum rotary evaporator. 0.80 g (92%) of 2-(2-butyl)-4-tert-butyl-6-(4,5-benzo-1,2,3-triazol-2-yl)-phenol (compound 103, Table 1), m.p. 81-84° C., is obtained.

### EXAMPLE 6

### Preparation of Compound 104 (Table 1)

1.0 g (3.89 mmol) of 4-methyl-2-(2-nitrophenylazo)-phenol (compound 206, Table 1) is stirred for 4 hours at 130° C. with 5 ml of N,N-dimethylacetamide and 0.30 g (4.66 mmol) of sodium azide. After the reaction has taken place, the mixture is taken up in water and extracted with toluene. The organic phases are washed five times with water and once with 2N hydrochloric acid solution, dried over sodium sulfate and concentrated by evaporation in vacuo at 80° C. 0.81 g (86%) of 4-methyl-2-(4,5-benzo-1,2,3-triazol-2-yl)-phenol (compound 104, Table 1), m.p. 128-132° C. (Lit. 131.5-133° C.; J. H. Hall, J. Org. Chem. 1986, 33, 2954).

The use of dimethyl sulfoxide, dimethylformamide or N-methylpyrrolidone instead of dimethylacetamide under otherwise identical conditions produces very similar results.

### EXAMPLE 7

### Preparation of Compound 105 (Table 1)

1.0 g (2.11 mmol) of 2-cumyl-4-(1,1,3,3-tetramethyl-butyl)-6-(2-nitrophenylazo)-phenol (compound 207, Table 2) is stirred for approximately 4 hours at 130° C. with 5 ml of N,N-dimethylacetamide and 0.165 g (2.53 mmol) of sodium azide. After the reaction has taken place, the mixture is taken up in water and extracted with toluene. The organic phases are washed five times with water and once with 2N hydrochloric acid solution, dried over sodium sulfate and concentrated using a vacuum rotary evaporator. 0.94 g (97%) of 2-cumyl-4-(1,1,3,3-tetramethyl-butyl-6-(4,5-benzo-1,2,3-triazol-2-yl)-phenol (compound 105, Table 1), m.p. 128-132° C., is obtained

### EXAMPLE 8

### Preparation of Compound 106 (Table 1)

58.5 g (0.15 mol) of 2,4-bis-(1,1-dimethylpropyl-)-6-(2-nitrophenylazo)-phenol (compound 208, Table 2) are stirred for 13 hours at 130° C. with 120 g of dimethylformamide and 10.8 g (0.164 mol) of sodium azide. After the reaction has taken place, dimethylformamide is distilled off in vacuo and the mixture is taken up in xylene. After the addition of water, the aqueous phase is separated off and the organic phase is dried over sodium sulfate and concentrated using a vacuum rotary evaporator. Crystallisation from methanol yields 49.5 g (93.9%) of 2,4-bis-(1,1-dimethylpropyl)-6-(4,5-benzo-1,2,3-triazol-2-yl)-phenol (compound 106, Table 1), m.p. 80-88° C.

### EXAMPLE 9

### Preparation of Compound 107 (Table 1)

a) Preparation of Compound 209 (Table 2).

25.25 9 (0.20 mol) of 2-chloroaniline are introduced in the course of 20 minutes at 20° C., with stirring, into 53.6 g of 32% hydrochloric acid, a suspension being formed. After the addition of 10 ml of water and cooling to from 10 to 0° C., 34.5 g of a 40% solution of sodium nitrite in water are added dropwise in the course of 30 minutes. The resulting diazonium salt solution is stirred for a further 30 minutes at −10° C. After the addition of 0.06 g of urea to eliminate excess nitrite, undissolved constituents are filtered off. The solution of the diazonium salt is so added in the course of 45 minutes, with cooling, to a solution of 6.96 g (0.174 mol) of sodium hydroxide and 41.15 g (0.174 mol) of β-(4-tert-butyl-4-hydroxyphenyl)-propionic acid methyl ester in 150 ml of methanol that the temperature remains below 0° C. When the addition of the diazonium salt is complete, the mixture is gradually brought to 25° C. and stirred for 90 minutes to complete the reaction. After the introduction of 250 ml of toluene with stirring, and the addition of 30 ml of water, the aqueous phase is separated off and the organic phase is washed with 100 ml of water. The organic phase is dried over sodium sulfate and concentrated using a vacuum rotary evaporator. Crystallisation of the residue from isopropanol yields 36.2 g (50%) of compound 209, (Table 2), m.p. 88-91° C.

b) Preparation of Compound 107 (Table 1).

9.0 g (24.0 mmol) of compound 209 (Table 2, prepared according to Example 9a) are stirred for 2 hours at 90° C. with 30 ml of dimethylformamide, 2.1 9 (32.0 mmol) of sodium azide and 36 mg of copper(I) bromide. After the reaction has taken place, the mixture is cooled, taken up in 50 ml of water and extracted with 50 ml of toluene. The organic phases are washed twice with water, once with 2N hydrochloric acid and once with saturated sodium chloride solution, dried over sodium sulfate and concentrated using a vacuum rotary evaporator. Crystallisation of the residue from toluene/methanol yields 6.41 g (76%) of compound 107, (Table 1), m.p. 119-124° C.

Analogously to Example 9b, compound 107 (Table 1) is likewise obtained using compound 213 (Table 2) instead of compound 209 (Table 2).

### EXAMPLE 10

### Preparation of Compound 108 (Table 1) with the Isolation of Compound 210 (Table 2)

a) Preparation of Compound 210 (Table 2).

39.12 g (0.2 mol) of 3-amino-4-chlorobenzotrifluoride are introduced in the course of 100 minutes at from 0 to 5° C., with stirring, into a mixture of 80 g of water and 54 g of 32% hydrochloric acid. After further stirring for 30 minutes at 0° C., 34.5 g (0.2 mol) of a 40% solution of sodium nitrite in water are added dropwise in the course of 75 minutes. The suspension is stirred for a further 45 minutes at 0° C. The resulting suspension of the diazonium salt is so added in the course of 45 minutes, with cooling, to a solution of 9.04 g (0.226 mol) of sodium hydroxide and 42.6 g (0.1244 mol) of 2-cumyl-4-(1,1,3,3-tetramethylbutyl)-phenol in 20 ml of xylene and 140 g of methanol that the temperature remains below 5° C. After half of the addition, a further 20 g of xylene and 140 g of methanol are added. When the addition of the diazonium salt is complete, the mixture is gradually brought to 25° C. and stirred for 18 hours to complete the reaction. After removal of the aqueous phase, the organic phase is neutralised with acetic acid and washed with 100 ml of water. The organic phase is dried over sodium sulfate and concentrated using a vacuum rotary evaporator. Crystallisation of the residue from isopropanol yields 42.3 g of 2-cumyl-4-(1,1,3,3-tetramethyl-butyl)-6-(2-chloro-5-trifluoromethyl-phenylazo)-phenol (compound 210, Table 2), m.p. 120-127° C.

b) Preparation of Compound 108 (Table 1).

9.0 g (16.9 mmol) of 2-cumyl-4-(1,1,3,3-tetramethyl-butyl)-6-(2-chloro-5-trifluoromethyl-phenylazo)-phenol (compound 210, Table 2, prepared according to Example 10a) are stirred for 2 hours at 120° C. with 30 ml of dimethylformamide, 1.7 9 (16 mmol) of triethylamine and 1.5 g (22.2 mmol) of sodium azide. After the reaction has taken place, the mixture is cooled, taken up in 50 ml of water and extracted with 50 ml of toluene. The organic phase is washed four times with water, dried over sodium sulfate and concentrated using a vacuum rotary evaporator. Crystallisation of the residue from isopropanol/methanol=3:1 yields 6.8 g (79%) of 2-cumyl-4-(1,1,3,3-tetramethyl-butyl)-6-[(5′-trifluoromethyl)-4,5-benzo-1,2,3-triazol-2-yl]-phenol (compound 108, Table 1), m.p. 92-95° C.

### EXAMPLE 11

### Preparation of Compound 108 (Table 1) Without the Isolation of Compound 210 (Table 2)

39.12 g (0.20 mol) of 3-amino-4-chlorobenzotrifluoride are diazotized as described in Example 10a, but at from 0 to −10° C. The resulting suspension is stirred for 70 minutes at from −5 to −10° C. The resulting suspension of the diazonium salt is so added in the course of 50 minutes, with-cooling, to a solution of 14.4 g (0.366 mol) of sodium hydroxide and 68.32 9 (0.20 mol) of 2-cumyl-4-(1,1,3,3-tetramethybutyl)-phenol in 30 ml of xylene and 170 ml of methanol that the temperature remains below −5° C. When the addition of the diazonium salt is complete, the mixture is gradually brought to 25° C. and is further stirred for 120 minutes. After removal of the aqueous phase, 200 ml of xylene are added and the organic phase is neutralised with acetic acid, washed with 100 ml of water and concentrated using a vacuum rotary evaporator. After concentration of the organic phase in vacuo, 118.6 g of the crude product of compound 210 (Table 2) are obtained. 117.6 9 of that crude product are heated to 135° C. in the course of 90 minutes with 300 ml of DMF, 22.4 g (0.22 mol) of triethylamine and 18.7 g (0.28 mol) of sodium azide and stirring is carried out for approximately 4.5 hours at from 130 to 135° C. After the reaction has taken place, the mixture is cooled, diluted with 500 ml of water and extracted with 500 ml of toluene. The organic phase is washed four times with water, once with 2N hydrochloric acid and once with saturated sodium chloride solution, dried over sodium sulfate and concentrated using a vacuum rotary evaporator. Crystallisation of the residue from xylene/methanol yields 58.9 g (58%) of 2-cumyl-4-(1,1,3,3-tetramethyl-butyl)-6-[(5′-trifluoromethyl)-4,5-benzo-1,2,3-triazol-2-yl]-phenol (compound 108, Table 1), m.p. 92-95° C.

### EXAMPLE 12

### Preparation of Compound 109 (Table 1) with the Isolation of Compound 211 (Table 2)

a) Preparation of Compound 211 (Table 2).

32.44 g (0.20 mol) of molten 2,5-dichloroaniline are introduced in the course of 40 minutes at 50° C., with stirring, into 53.6 g of 32% hydrochloric acid. After the addition of 20 ml of water and cooling to 0° C., 34.5 g of a 40% solution of sodium nitrite in water are added dropwise in the course of 80 minutes. The suspension is stirred at 0° C. for 85 minutes. The resulting suspension of the diazonium salt is so added in the course of 50 minutes, with cooling, to a solution of 9.04 g (0.226 mol) of sodium hydroxide and 25.7 g (0.1244 mol) of 2,4-di-tert-butyl-phenol in 150 ml of methanol that the temperature remains below 0° C. When the addition of the diazonium salt is complete, the mixture is gradually brought to 25° C. and further stirred for 90 minutes. After the addition of 400 ml of toluene, the aqueous phase is separated off and the organic phase is neutralised with acetic acid, washed with water, dried over sodium sulfate and concentrated using a vacuum rotary evaporator. Crystallisation of the residue from a 1:1 mixture of xylene/methanol yields 28.1 9 (64%) of 2,4-di-tert-butyl-6-(2,5-dichlorophenylazo)-phenol (compound 211, Table 2), m.p. 121-126° C.

b) Preparation of Compound 109 (Table 1).

9.0 g (23.7 mmol) of 2,4-di-tert-butyl-6-(2,5-dichlorophenylazo)-phenol (compound 211, Table 2, prepared according to Example 12a) are stirred for 4 hours at 80° C. with 30 ml of dimethylformamide, 2.0 g (30.8 mmol) of sodium azide, 2.43 g of triethylamine and 0.034 g of copper(I) bromide. After the reaction has taken place, the mixture is taken up in water and extracted with toluene. The organic phases are washed four times with water, once with 2N hydrochloric acid and once with saturated sodium chloride solution, dried over sodium sulfate and concentrated using a vacuum rotary evaporator. Crystallisation of the residue from toluene/methanol yields 7.13 g (84%) of 2,4-di-tert-butyl-6-(5′-chloro-4,5-benzo-1,2,3-triazol-2-yl)-phenol (compound 109, Table 1), m.p. 149-153° C.

### EXAMPLE 13

### Preparation of Compound 109 (Table 1) with the Isolation of Compound 212 (Table 2)

a) Preparation of Compound 212 (Table 2).

32.44 g (0.20 mol) of molten 2,4-dichloroaniline are introduced in the course of 40 minutes at 50° C., with stirring, into 53.6 g of 32% hydrochloric acid. After the addition of 40 ml of water and cooling to 0° C., 34.5 g of a 40% solution of sodium nitrite in water are added dropwise in the course of 65 minutes. The suspension is stirred for 85 minutes at 0° C. The resulting suspension of the diazonium salt is so added in the course of 85 minutes, with cooling, to a solution of 9.04 g (0.226 mol) of sodium hydroxide and 25.7 g (0.1244 mol) of 2,4-di-tert-butyl-phenol in 150 ml of methanol that the temperature remains below 0° C. When the addition of the diazonium salt is complete, the mixture is gradually brought to 25° C. and stirred for a further 90 minutes. After decanting off the liquid phase, the solid product is taken up in 200 ml of toluene and washed with 100 ml of water. The organic phase is dried over sodium sulfate and concentrated using a vacuum rotary evaporator. Crystallisation of the residue from a 1:1 mixture of xylene/methanol yields 31.3 g (70%) of 2,4-di-tert-butyl-6-(2,4-dichlorophenylazo)-phenol (compound 212, Table 2), m.p.164-168° C.

b) Preparation of Compound 109, Table 1).

9.0 g (23.7 mmol) of 2,4-di-tert-butyl-6-(2,4-dichlorophenylazo)-phenol (compound 212, Table 2, prepared according to Example 13a), are reacted with sodium azide as described in Example 12b. Crystallisation of the residue from toluene/methanol yields 6.55 g (77%) of 2,4-di-tert-butyl-6-(5′-chloro-4,5-benzo-1,2,3-triazol-2-yl)-phenol (compound 109, Table 1), m.p. 149-153° C.

### EXAMPLE 14

### Preparation of Compound 110 (Table 1)

a) Preparation of Compound 214 (Table 2).

10.8 g (63 mmol) of 2-chloro-5-nitroaniline are triturated with 0.2 g of Steramid [N,N′-ethylene-bis-stearic amide; C.A. Reg. No. 110-30-5]. The mixture is then introduced in portions into a solution of 16.5 ml of concentrated hydrochloric acid in 10 ml of water and stirring is carried out for 16 hours. After cooling to from −10 to −15° C., 16 ml of a 4N solution of sodium nitrite in water are added dropwise in the course of from 1 to 2 hours. The resulting diazonium salt solution is stirred for 10 minutes at −10° C. 2.2 g (54 mmol) of sodium hydroxide beads are dissolved, with stirring, in a solution of 18.5 g (54 mmol) of 4-(1,1,3,3 tetramethylbutyl)-2-cumyl-phenol (95% purity) in 140 ml of methanol and 20 ml of xylene. 4.0 g (54 mmol) of calcium hydroxide are then added and the resulting suspension is cooled to lower −15° C. Then, at from −15 to −5° C., the diazonium salt solution is added dropwise within a period of 30 minutes. The red suspension is gradually brought to 25° C. and stirred overnight to complete the reaction. After the introduction of 150 ml of toluene with stirring and the addition of 100 ml of water, the aqueous phase is separated off and the organic phase is washed four times with 100 ml of water each time. The organic phases are combined and concentrated using a vacuum rotary evaporator. Crystallisation of the residue from xylene/-methanol yields 16.2 g (59%) of 2-(2-chloro-5-nitrophenylazo)-6-(1-methyl-1-phenylethyl)-4-(1,1,3,3-tetramethylbutyl)-phenol (compound 214, Table 2). M.p. 153-156° C.

b) Preparation of Compound 110 (Table 1).

10.16 g (20 mmol) of 2-(2-chloro-5-nitro-phenylazo)-6-(1-methyl-1-phenyl-ethyl)-4-(1,1,3,3-tetramethyl-butyl)-phenol [compound 214 (Table 2), prepared according to Example 14a], are stirred for approximately 4 hours at 40° C. with 60 ml of dimethylformamide and 1.69 g (26 mmol) of sodium azide. After the reaction has taken place, the mixture is taken up in water and extracted with methylene chloride. The organic phases are washed repeatedly with water and concentrated by evaporation. Crystallisation of the residue from isopropanol/toluene yields 8.53 g (88%) of 2-(1-methyl-1-phenyl-ethyl)-6-(5-nitro-benzotriazol-2-yl)-4-(1,1,3,3-tetramethyl-butyl)-phenol (compound 110, Table 1). M.p. 153-156° C.

### EXAMPLE 15

### Preparation of Compound 111 (Table 1)

a) Preparation of Compound 215 (Table 2).

45 g of 32% hydrochloric acid are rapidly added to a solution of 11.3 g (66 mmol) of 3-amino-4-chlorobenzoic acid in 10.9 g of 30% sodium hydroxide solution and 40 ml of water. After subsequently stirring for one hour, the mixture is cooled to from −5 to 0° C. 12.5 ml of an aqueous 40% sodium nitrite solution (approximately 65 mmol of NaNO₂) are then metered in at from −5 to 0° C. After subsequently stirring for one hour, excess nitrite is eliminated using a small amount of sulfamic acid. 3.04 g (76 mmol) of sodium hydroxide beads are dissolved, with stirring, in a solution of 26.0 g (76 mmol) of 4-(1,1,3,3-tetramethylbutyl)-2-cumyl-phenol (95% purity) in 70 ml of methanol and 10 ml of xylene. 5.63 g (76 mmol) of calcium hydroxide are then added and the resulting suspension is cooled to 0° C. The diazonium salt solution is then added dropwise thereto at from 0 to 5° C. In parallel, approximately 10 g of calcium hydroxide and 20 ml of 30% sodium hydroxide solution are metered in in order to maintain an alkaline pH. When the addition is complete, the mixture is gradually brought to 25° C. and stirred overnight to complete the reaction. After the introduction, with stirring, of 50 ml of water, 50 ml of 32% hydrochloric acid, 100 ml of toluene and 200 ml of ethyl acetate, the aqueous phase is separated off and the organic phase is washed twice with 100 ml of water. The organic phases are combined and concentrated using a vacuum rotary evaporator. Crystallisation of the residue from methanol/hexane yields 12.1 g (36%) of 4-chloro-3-[2-hydroxy-3-(1-methyl-1-phenyl-ethyl)-5-(1,1,3,3-tetramethyl-butyl)-phenylazo]-benzoic acid (compound 215, Table 2). M.p. 218-220° C.

b) Preparation of Compound 111 (Table 1).

5.07 g (10 mmol) of 4-chloro-3-[2-hydroxy-3-(1-methyl-1-phenyl-ethyl)-5-(1,1,3,3-tetramethyl-butyl)-phenylazo]-benzoic acid [compound 215 (Table 2), prepared according to Example 15a] are stirred for approximately 4 hours at 140° C. with 35 ml of dimethylformamide and 0.85 g (13 mmol) of sodium azide. After the reaction has taken place, the mixture is taken up in water, 5 ml of acetic acid are added and extraction is carried out with toluene. The organic phases are washed repeatedly with water and concentrated using a vacuum rotary evaporator. Crystallisation of the residue from isopropanol yields 2.5 g (52%) of 2-[2-hydroxy-3-(1-methyl-1-phenyl-ethyl)-5-(1,1,3,3-tetramethyl-butyl)-phenyl]-2H-benzotriazole-5-carboxylic acid (compound 111, Table 1). M.p. 219-221° C.

### EXAMPLE 16

### Preparation of Compound 112 (Table 1)

a) Preparation of Compound 216 (Table 2).

5.0 g (32.7 mmol) of 3-amino-4-chlorobenzonitrile are stirred, in portions, into 60 ml of water at 95° C. 30 ml of 32% hydrochloric acid are then added dropwise, and the mixture is cooled to room temperature and stirred for 16 hours to complete the reaction. After cooling to from −10 to −15° C., 9.0 ml of a 4N solution of sodium nitrite in water are metered in in the course of 40 minutes. The resulting diazonium salt solution is stirred for 30 minutes at −10° C. 2.2 g (54 mmol) of sodium hydroxide beads are dissolved, with stirring, in a solution of 5 g (0.54 mmol) of 4-(1,1,3,3 tetramethylbutyl)-2-cumyl-phenol (95% purity) in 70 ml of methanol and 10 ml of xylene. After the solution has been cooled to <−15° C., the diazonium salt solution is added dropwise thereto within a period of 100 minutes at from −15 to −5° C. During the addition an alkaline pH is maintained by metering in approximately 30 ml of 30% sodium hydroxide solution in parallel. When the addition is complete, 50 ml of xylene are added. The red suspension is gradually brought to 25° C. and stirred overnight to complete the reaction. After the introduction of 150 ml of ethyl acetate with stirring, and the addition of 100 ml of water and 5 ml of acetic acid, the aqueous phase is separated off and the organic phase is washed three times with 100 ml of water each time. The organic phases are combined and concentrated using a vacuum rotary evaporator. Crystallisation of the residue from methanol yields 9.4 g (59%) of 4-chloro-3-[2-hydroxy-3-(1-methyl-1-phenyl-ethyl)-5-(1,1,3,3-tetramethyl-butyl)-phenylazo]-benzonitrile (compound 216, Table 2). M.p. 190-191° C.

b) Preparation of Compound 112 (Table 1).

4.88 g (10 mmol) of 4-chloro-3-[2-hydroxy-3-(1-methyl-1-phenyl-ethyl)-5-(1,1,3,3-tetramethyl-butyl)-phenylazo]-benzonitrile [compound 216 (Table 2), prepared according to Example 16a] are stirred for one hour at 120° C. with 12.5 ml of dimethylformamide and 0.85 g (13 mmol) of sodium azide. After the reaction has taken place, the mixture is taken up in water and extracted with toluene. The organic phases are washed repeatedly with water, combined and concentrated using a vacuum rotary evaporator. Crystallisation of the residue from hexane yields 3.6 g (77%) of 2-[2-hydroxy-3-(1-methyl-1-phenyl-ethyl)-5-(1,1,3,3-tetramethyl-butyl)-phenyl]-2-benzotriazole-5-carbonitrile (compound 112, Table 1). M.p. 199-201° C.

### EXAMPLE 17

### Preparation of Compound 113 (Table 1)

a) Preparation of Compound 218 (Table 2).

27.4 g (0.132 mol) of 2-chloroaniline-5-sulfonic acid are dissolved in 21.8 g of 30% sodium hydroxide solution and 80 ml of water. 90 ml of 32% hydrochloric acid are then rapidly added and the suspension is stirred for one hour. After cooling to from 0 to −5° C., 32.5 ml of a 4N solution of sodium nitrite in water are metered in in the course of 60 minutes. After subsequently stirring for one hour at from 0 to -5° C., excess nitrite is eliminated using a small amount of sulfamic acid. 6.08 g (0.152 mol) of sodium hydroxide beads are dissolved, with stirring, in a solution of 45.1 g (0.132 mol) of 4-(1,1,3,3 tetramethylbutyl)-2-cumyl-phenol (95% purity) in 14 ml of methanol and 20 ml of xylene. 11.3 g (0.152 mol) of calcium hydroxide are then added and the resulting suspension is cooled to 0° C. The diazonium salt solution is then added dropwise thereto at from 0 to 5° C. In parallel, approximately 10 g of calcium hydroxide and 30 ml of 30% sodium hydroxide solution are metered in to maintain an alkaline pH. When the addition is complete, the mixture is gradually brought to 25° C. and stirred overnight to complete the reaction. After the addition of 100 ml of water and 75 ml of 32% hydrochloric acid, 300 ml of xylene and 150 ml of ethyl acetate are stirred in. The aqueous phase is separated off and the organic phase is washed three times with 100 ml of water each time. The organic phases are combined and concentrated using a vacuum rotary evaporator. 68.3 g of crude 4-chloro-3-[2-hydroxy-3-(1-methyl-1-phenyl-ethyl)-5-(1,1,3,3-tetramethyl-butyl)-phenylazo]-benzenesulfonic acid (compound 217, Table 2) are obtained. M.p. 182° C. (decomposition). 300 ml of water and 30 ml of 30% sodium hydroxide solution are added to 67.3 g (0.124 mol) of that compound, and the mixture is heated to from 80 to 85° C. and stirred for 0.5 hour at that temperature. After cooling to room temperature, the supernatant aqueous phase is decanted off and the residue is dissolved hot in 150 ml of water and 150 ml of ethanol. The product that crystallises out on cooling is dried in vacuo. 48.0 g (64%) of 4-chloro-3-[2-hydroxy-3-(1-methyl-1-phenyl-ethyl)-5-(1,1,3,3-tetramethyl-butyl)-phenylazo]-benzenesulfonic acid sodium salt (compound 218, Table 2) are obtained. M.p. 243-245° C.

b) Preparation of Compound 113 (Table 1).

5.65 g (10 mmol) of 4-chloro-3-[2-hydroxy-3-(1-methyl-1-phenyl-ethyl)-5-(1,1,3,3-tetramethyl-butyl)-phenylazo]-benzenesulfonic acid sodium salt [compound 218 (Table 2), prepared according to Example 17a] are stirred for 8 hours at 160° C. with 20 ml of N-methyl-2-pyrrolidone and 0.85 g (13 mmol) of sodium azide. After the reaction has taken place, 50 ml of toluene, 30 ml of water and three drops of 2N hydrochloric acid are added. The phases are separated and the organic phase is washed with 30 ml of water. The aqueous phases are re-extracted with 30 ml of toluene. The organic phases are combined and concentrated using a vacuum rotary evaporator. Crystallisation of the residue from isopropanol yields 3.9 g (71%) of 2-[2-hydroxy-3-(1-methyl-1-phenyl-ethyl)-5-(1,1,3,3-tetramethyl-butyl)-phenyl]-2H-benzotriazole-5-sodium sulfonate (compound 113, Table 1). M.p. 361° C. (decomposition).

### EXAMPLE 18

### Preparation of Compound 114 (Table 1)

0.30 g (0.415 mmol) of 2,2′-methylene-bis[6-(2-nitrophenyl)azo-4-(1,1,3,3-tetramethylbutyl)-phenol] is stirred for 8 hours at 160° C. with 0.5 ml of N-methyl-2-pyrrolidone and 0.072 g (1.1 05 mmol) of sodium azide. After the reaction has taken place, according to HPLC analysis the reaction mass contains 24% by weight of 2,2-methylenebis[6-(2H-benzotriazol-2-yl)-4-(1,1,3,3-tetramethylbutyl)-phenol (compound 114, Table 1, corresponding to Tinuvin 360 (RTM), Ciba Spezialitatenchemie AG).

### EXAMPLE 19

### Preparation of a Mixture of Compounds 115, 116, 107 (Table 1)

43.26 g (115 mmol) of 3-(1,1-dimethylethyl)-4-hydroxy-5-[(2-chlorophenyl)-azo]-benzene-propanoic acid methyl ester [compound 209 (Table 2), prepared according to Example 9a] is stirred for 30 minutes at 150° C. and then for 13 hours at from 160 to 170° C. with 36.0 g (120 mmol) of polyethylene glycol 300, 9.75 g (150 mmol) of sodium azide, 0.5 ml of triethylamine and 85.6 mg (1.15 mmol) of CuBr, during which methanol is continuously distilled off. The mixture is then evacuated to 200 mbar and maintained for a further 2 hours at from 160 to 170° C. After the reaction has taken place, according to HPLC analysis the liquid component of the reaction mass contains 56.7% of compound 115 (Table 1), 30.4% of compound 116 (Table 1) and 1.5% of compound 107 (Table 1).

### EXAMPLE 20

### Preparation of Compound 117 (Table 1)

25 ml of dimethylformamide, 5 g (38.4 mmol) of isooctanol (isomeric mixture) and 1.35 g (20.8 mmol) of sodium azide are added to 6.12 g (16 mmol) of 3-(1,1-dimethylethyl)-4-hydroxy-5-[(2-nitrophenyl)-azo]-benzenepropanoic acid methyl ester [compound 213, Table 2, prepared analogously to Example 9a] and the mixture is stirred at 150° C. Methanol/DMF is distilled off at the outset at normal pressure and after 5 hours at slightly reduced pressure. After 6 hours, a further 2.5 g (19.2 mmol) of isooctanol is added and the temperature is tained at 150° C. for a further 5.5 hours. After the reaction has taken place, according to GC analysis the liquid components of the reaction mass contain up to 35% of compound 117 (Table 1).

TABLE 1

| Compound | Structural formula |
|---|---|
| 101 | 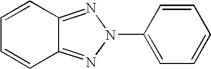 |
| 102 | 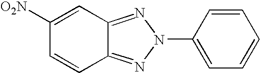 |
| 103 | 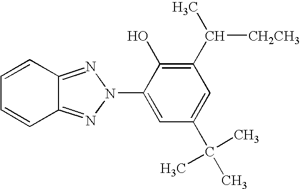 |
| 104 | 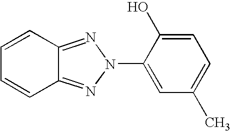 |
| 105 | 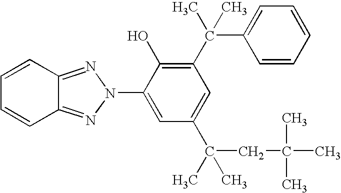 |
| 106 | 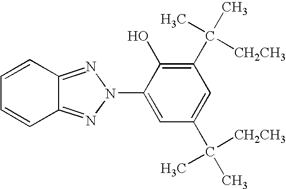 |
| 107 | 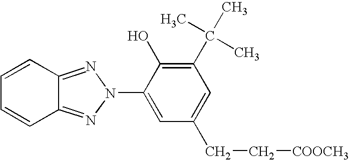 |
| 108 | 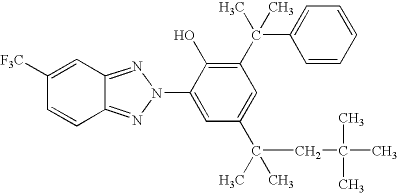 |
| 109 | 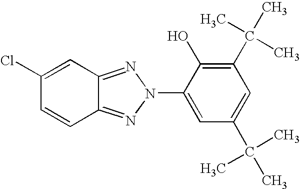 |
| 110 | 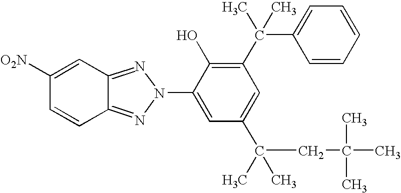 |
| 111 | 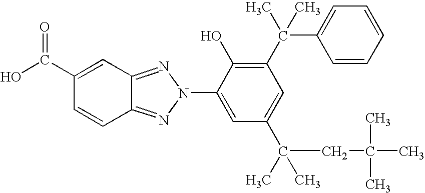 |
| 112 | 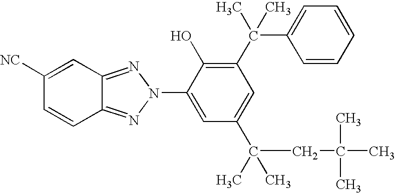 |
| 113 | 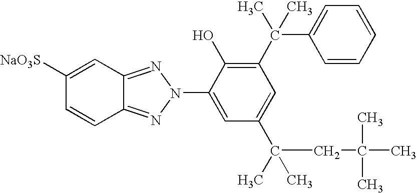 |
| 114 | 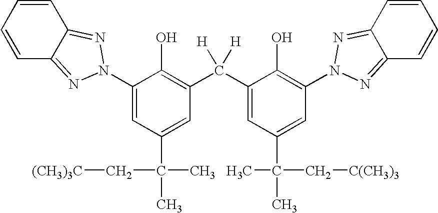 |
| 115 | 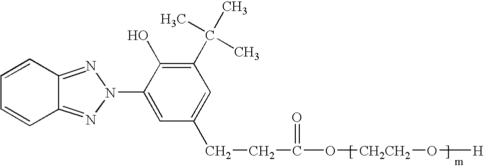 |
| 116 | 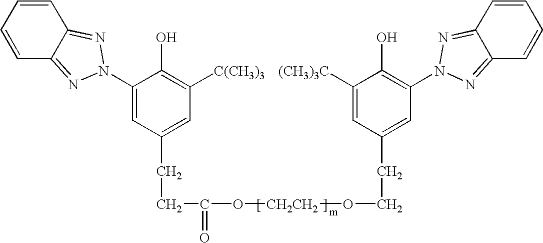 |
| 117 | 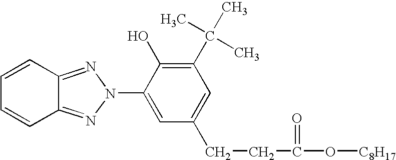 |

TABLE 2

| Compound | Structural formula |
|---|---|
| 201 | 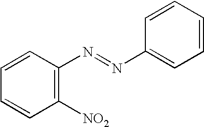 |
| 202 | 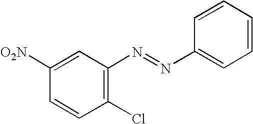 |
| 203 | 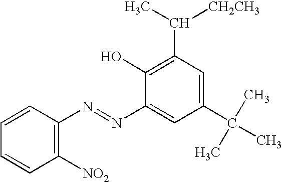 |
| 204 | 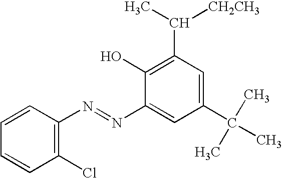 |
| 205 | 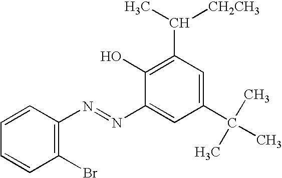 |
| 206 | 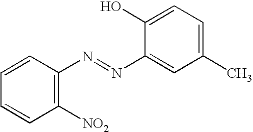 |
| 207 | 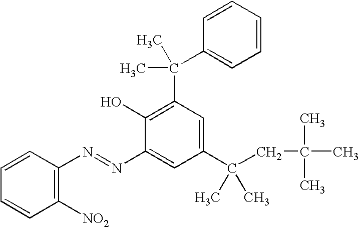 |
| 208 | 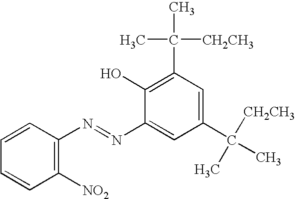 |
| 209 | 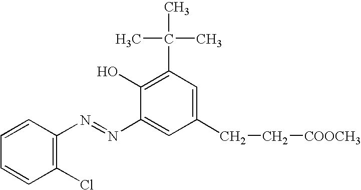 |
| 210 | 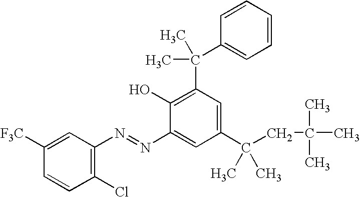 |
| 211 | 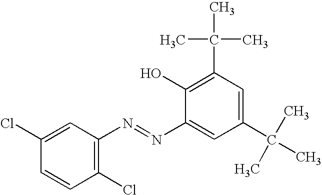 |
| 212 | 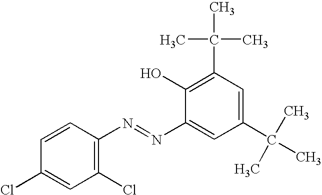 |
| 213 | 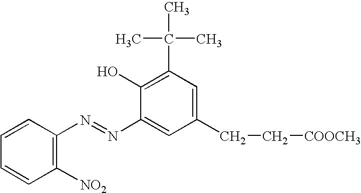 |
| 214 | 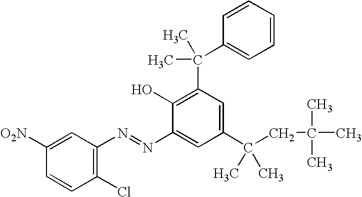 |
| 215 | 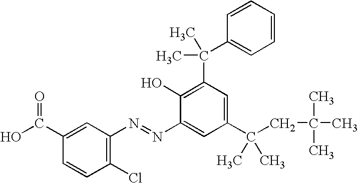 |
| 216 | 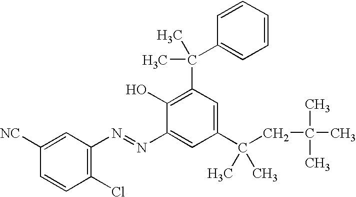 |
| 217 | 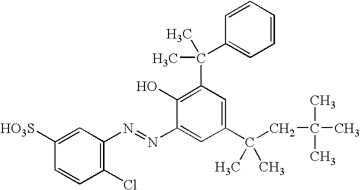 |
| 218 | 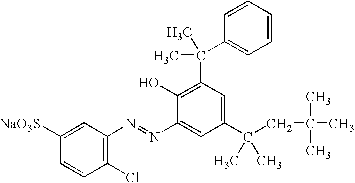 |
| 219 | 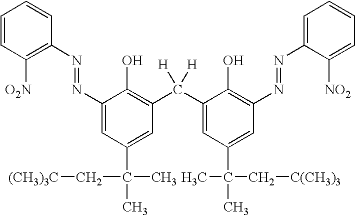 |

What is claimed is:

1. A process for the preparation of a compound of formula I 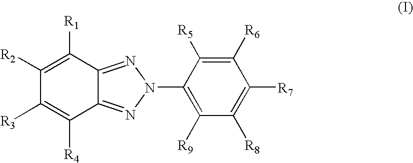
wherein
R₅ is hydroxy,
R₁, R₂, R₃, R₄, R₆, R₇, R₈ and R₉ are each independently of the others hydrogen, halogen, —SO₃H, —SO₃⁻Mₐ⁺, hydroxy, carboxy, cyano, nitro, C₁-C₂₅alkyl, C₁-C₂₅alkyl substituted by halogen, hydroxy, carboxy, cyano, C₁-C₁₈alkoxycarbonyl, C₁-C₄alkoxy or by amino; C₂-C₂₄-alkenyl, C₇-C₉phenylalkyl, unsubstituted or C₁-C₄alkyl-substituted phenyl, unsubstituted or C₁-C₄alkyl-substituted C₅-C₈cycloalkyl; C₁-C₁₈alkoxy, C₁-C₁₈alkylthio, C₁-C₁₈alkylsulfonyl, unsubstituted or C₁-C₄alkyl-substituted phenylsulfonyl, unsubstituted or C₁-C₄alkyl-substituted phenylthio; amino, C₁-C₄alkylamino, di(C₁-C₄alkyl)amino, C₁-C₂₅alkanoyl, C₁-C₂₅alkoxycarbonyl, C₁-C₂₅alkanoyloxy,
C₁-C₂₅alkanoylamino, C₃-C₂₅alkyl interrupted by oxygen, sulfur or by 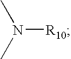
C₃-C₂₅alkoxy interrupted by oxygen, sulfur or by 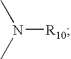
C₃-C₂₅alkanoyloxy interrupted by oxygen, sulfur or by 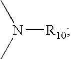
C₃-C₂₅alkoxycarbonyl interrupted by oxygen, sulfur or by 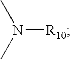
C₆-C₉cycloalkoxycarbonyl, C₆-C₉cycloalkylcarbonyloxy, unsubstituted or C₁-C₁₂alkyl-substituted benzoyloxy; —(CH₂)ₚ—COR₁₁ or —(CH₂)_{q}OH, or, further, the radicals R₆ and R₇ or the radicals R₇ and R₈ or the radicals R₈ and R₉, together with the carbon atoms to which they are bonded, form a benzo ring, R₃ in addition is a radical of formula II 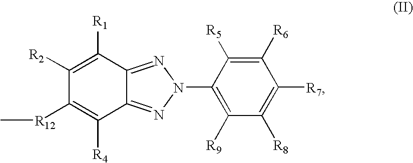
and R₆ in addition is a radical of formula III 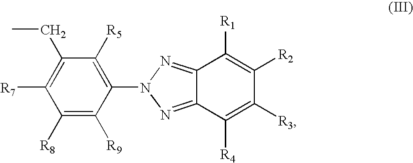
R₁₀ is hydrogen or C₁-C₈alkyl,
R₁₁ is hydroxy,
$$[ {—O}^{-} \frac{1}{r} {M}_{b}^{r +} ] ,$$
C₁-C₁₈alkoxy, 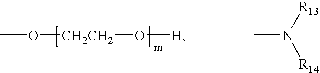
or a radical of formula IV 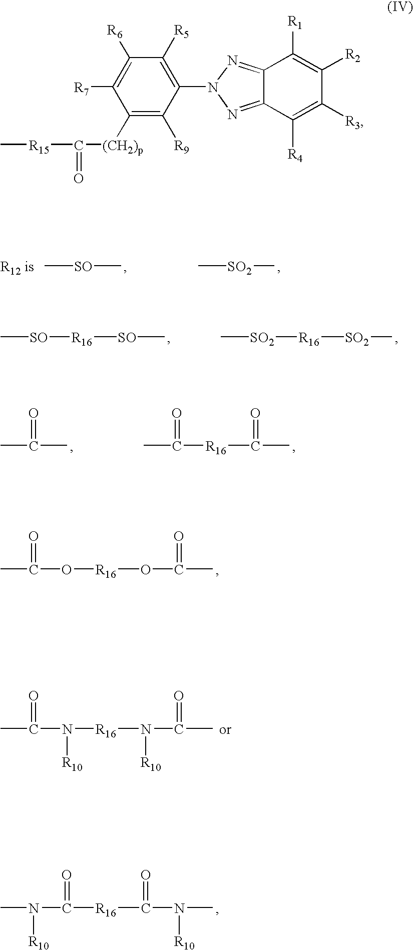
R₁₃ and R₁₄ are each independently of the other hydrogen or C₁-C₁₈alkyl,
R₁₅ is —O—R₁₇—O—,
R₁₆ is C₂-C₁₂alkylene, C₅-C₁₂cycloalkylene, or C₈-C₁₂alkylene interrupted or terminated by cyclohexylene;
R₁₇ is C₂-C₁₂alkylene, or C₄-C₁₂alkylene interrupted by oxygen, sulfur or by 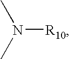
Mₐ is a monovalent metal cation,
M_{b} is an r-valent metal cation,
m is an integer from 1 to 20,
p is 0, 1 or 2,
q is 1, 2, 3, 4, 5 or 6, and
r is 1, 2 or 3,
which process comprises reacting a compound of formula V 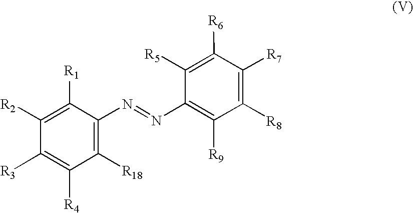
wherein
R₁, R₂, R₃, R₄, R₅, R₆, R₇, R₈ and R₉ are as defined hereinbefore, R₃ in addition being radical of formula VI 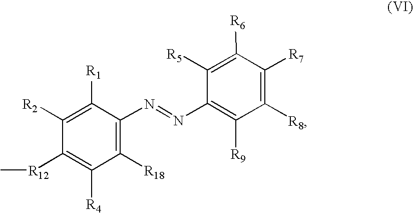
R₆ in addition being a radical of formula VII 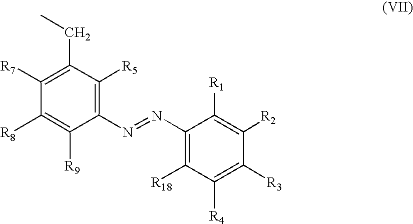
and R₁₁ in addition being a radical of formula VIII 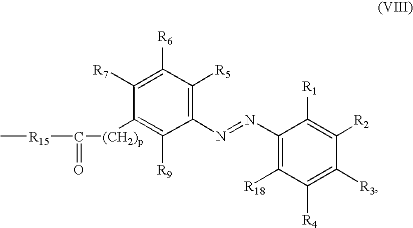
R₁₈ is halogen, nitro, 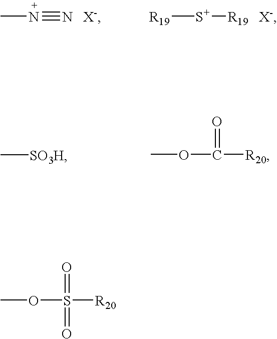
or C₁-C₁₈alkoxy,
R₁₉ is C₁-C₁₈alkyl, unsubstituted or C₁-C₄alkyl-substituted phenyl; or unsubstituted or C₁-C₄alkyl-substituted C₅-C₈cycloalkyl,
R₂₀ is C₁-C₁₈alkyl, unsubstituted or C₁-C₄alkyl-, halo- or nitro-substituted phenyl; unsubstituted or
C₁-C₄alkyl-substituted C₅-C₈cycloalkyl; or fluorine-substituted C₁-C₁₈alkyl, and
X⁻ is chloride, bromide, iodide, hydroxide, nitrate or nitrite, with an azide compound of formula IX 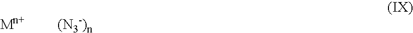
wherein
M is an n-valent metal cation, 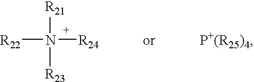
R₂₁, R₂₂, R₂₃ and R₂₄ are each independently of the others hydrogen or C₁-C₁₈alkyl,
R₂₅ is C₁-C₁₈alkyl, and
n is 1, 2or 3.

2. A process according to claim 1, wherein R₁₁ is hydroxy, 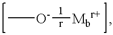
C₁-C₁₈alkoxy, 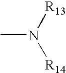
or a radical of formula IV;
R₁₃ and R₁₄ are each independently of the other hydrogen or C₁-C₁₈alkyl,
M_{b} is an r-valent metal cation, and
r is 1, 2 or 3.

3. A process according to claim 1, wherein
R₅ is hydroxy,
R₁, R₂, R₃, R₄, R₆, R₇, R₈ and R₉ are each independently of the others hydrogen, halogen,
—SO₃H,
—SO₃⁻Mₐ⁺, hydroxy, carboxy, cyano, nitro, C₁-C₁₈alkyl, fluorine- or C₁-C₄alkoxy-substituted C₁-C₁₈alkyl; C₂-C₁₈alkenyl, C₇-C₉phenylalkyl, unsubstituted or C₁-C₄alkyl-substituted phenyl;
C₅-C₈cycloalkyl,
C₁-C₁₈alkoxy, C₁-C₁₈alkylthio, C₁-C₁₈alkylsulfonyl, phenylsulfonyl, phenylthio,
C₁-C₄alkylamino,
di(C₁-C₄alkyl)amino, C₁-C₁₈alkanoyl, C₁-C₁₈alkoxycarbonyl, C₁-C₁₈alkanoyloxy, C₁-C₁₈alkanoylamino, C₃-C₁₈alkyl interrupted by oxygen, sulfur or by 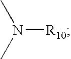
C₃-C₁₈alkoxy interrupted by oxygen, sulfur or by 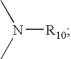
C₃-C₁₈alkanoyloxy interrupted by oxygen, sulfur or by 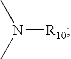
C₃-C₁₈alkoxycarbonyl interrupted by oxygen, sulfur or by 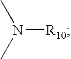
C₆-C₉cycloalkoxycarbonyl,
C₆-C₉cycloalkylcarbonyloxy, unsubstituted or C₁-C₄alkyl-substituted benzoyloxy; —(CH₂)ₚ—COR₁₁ or —(CH₂)_{q}OH, or, further, the radicals R₆ and R₇ or the radicals R₇ and R₈ or the radicals R₈ and R₉, together with the carbon atoms to which they are bonded, form a benzo ring, R₃ in addition is a radical of formula II and R₆ in addition is a radical of formula III, R₁₀ is hydrogen or C₁-C₆alkyl,
R₁₁ is hydroxy, 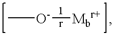
C₁-C₁₂alkoxy, 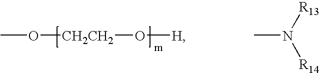
or a radical of formula IV,
R₁₂ is —SO₂—, —SO₂—R₁₆—SO₂—, 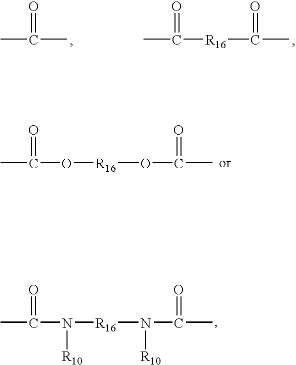
R₁₃ and R₁₄ are each independently of the other hydrogen or C₁-C₈alkyl,
R₁₅ is —O—R₁₇—O—,
R₁₆ is C₂-C₁₂alkylene or C₅-C₁₂cycloalkylene,
R₁₇ is C₂-C₈alkylene, or C₄-C₁₂alkylene interrupted by oxygen or by sulfur,
R₁₈ is halogen, nitro, 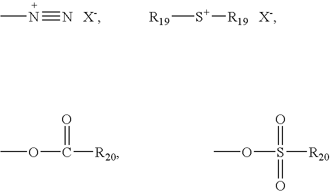
or
C₁-C₁₂alkoxy,
R₁₉ is C₁-C₁₈alkyl, phenyl or C₅-C₈cycloalkyl,
R₂₀ is C₁-C₁₈alkyl, unsubstituted or C₁-C₄alkyl-, fluorine-, chlorine- or nitro-substituted phenyl;
C₅-C₈cycloalkyl, or fluorine-substituted C₁-C₁₂alkyl,
R₂₁, R₂₂, R₂₃ and R₂₄ are each independently of the others hydrogen or C₁-C₁₂alkyl,
R₂₅ is C₁-C₁₂alkyl,
M is lithium, sodium, potassium, calcium, 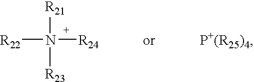
Mₐ is sodium or potassium,
M_{b} is sodium, potassium or calcium,
X⁻ is chloride or bromide,
m is an integer from 1 to 15,
n is 1 or 2,
p is 0, 1 or 2,
q is 1, 2 or 3, and
r is 1 or 2.

4. A process according to claim 1, wherein
R₁ is hydrogen, chlorine, carboxy, nitro, C₁-C₄alkyl, trifluoromethyl or C₁-C₄alkoxy,
R₂ is hydrogen, chlorine, —SO₃H, —SO₃⁻Mₐ⁺, carboxy, cyano, nitro, C₁-C₄alkyl, trifluoromethyl,
C₁-C₄alkoxy, benzyl, phenyl, cyclohexyl, C₁-C₄alkylsulfonyl, phenylsulfonyl, C₁-C₈alkanoyl,
C₁-C₈alkoxycarbonyl, C₁-C₈alkanoyloxy, C₃-C₈alkyl interrupted by oxygen or by sulfur; C₃-C₈-alkoxy interrupted by oxygen or by sulfur; C₃-C₈alkanoyloxy interrupted by oxygen or by sulfur; C₃-C₈alkoxycarbonyl interrupted by oxygen or by sulfur; cyclohexyloxycarbonyl, cyclohexylcarbonyloxy, or benzoyloxy,
R₃ is hydrogen or a radical of formula II,
R₄ is hydrogen, chlorine, carboxy, nitro, C₁-C₄alkyl, trifluoromethyl or C₁-C₄alkoxy,
R₆ is hydrogen, C₁-C₁₂alkyl, C₇-C₉phenylalkyl, phenyl, cyclohexyl, C₁-C₁₂alkoxy, C₃-C₁₂alkyl interrupted by oxygen or by sulfur; C₃-C₁₂alkoxy interrupted by oxygen or by sulfur; or a radical of formula III,
R₇ is hydrogen, chlorine, C₁-C₄alkyl or C₁-C₄alkoxy,
R₈ is hydrogen, C₁-C₁₂alkyl, C₇-C₉phenylalkyl, phenyl, cyclohexyl, C₁-C₁₂alkoxy, C₃-C₁₂alkyl interrupted by oxygen or by sulfur; C₃-C₁₂alkoxy interrupted by oxygen or by sulfur; or
—(CH₂)ₚ—COR₁₁,
R₉ is hydrogen, chlorine, C₁-C₄alkyl or C₁-C₄alkoxy,
R₁₁ is hydroxy, C₁-C₈alkoxy, 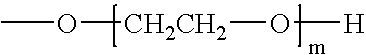
or a radical of formula IV,
R₁₂ is —SO₂—, 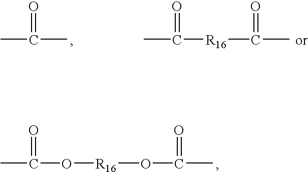
R₁₅ is —O—R₁₇—O—,
R₁₆ is C₂-C₁₈alkylene or C₅-C₈cycloalkylene,
R₁₇ is C₂-C₈alkylene, or C₄-C₁₂alkylene interrupted by oxygen,
R₁₈ is chlorine, bromine, iodine, nitro, 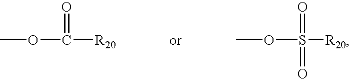
R₂₀ is C₁-C₈alkyl, unsubstituted or fluorine-, chlorine- or nitro-substituted phenyl; or fluorine-substituted C₁-C₄alkyl,
M is lithium, sodium, potassium or calcium,
Mₐ⁺ is sodium or potassium,
m is an integer from 1 to 15,
n is 1 or 2, and
p is 1 or 2.

5. A process according to claim 1, wherein R₁, R₄, R₇ and R₉ are hydrogen.

6. A process according to claim 1, wherein R₁₈ is nitro, chlorine or bromine.

7. A process according to claim 1, wherein
R₁ is hydrogen or C₁-C₄alkyl,
R₂ is hydrogen, chlorine, —SO₃H, —SO₃⁻Mₐ⁺, carboxy, cyano, nitro, C₁-C₄alkyl, trifluoromethyl,
C₁-C₄alkoxy, C₁-C₄alkanoyl, C₁-C₄alkoxycarbonyl, C₃-C₈alkyl interrupted by oxygen; or C₃-C₈-alkoxy interrupted by oxygen,
R₃ is hydrogen,
R₄ is hydrogen or C₁-C₄alkyl,
R₆ is hydrogen, C₁-C₈alkyl, C₇-C₉phenylalkyl, phenyl, cyclohexyl or a radical of formula III,
R₇ is hydrogen or C₁-C₄alkyl,
R₈ is hydrogen, C₁-C₁₂alkyl, C₇-C₉phenylalkyl, phenyl, cyclohexyl or —(CH₂)ₚ—COR₁₁,
R₉ is hydrogen or C₁-C₄alkyl,
R₁₁ is hydroxy, C₁-C₈alkoxy, 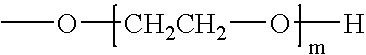
or a radical of formula IV,
R₁₅ is —O—R₁₇—O—,
R₁₇ is C₄-C₁₂alkylene interrupted by oxygen,
R₁₈ is chlorine, bromine, nitro, 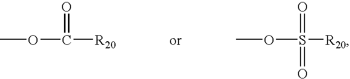
R₂₀ is C₁-C₄alkyl, unsubstituted or fluorine-, chlorine- or nitro-substituted phenyl; or trifluoromethyl,
M is lithium, sodium or potassium,
Mₐ⁺ is sodium or potassium,
m is an integer from 1 to 10,
n is 1, and
p is 2.

8. A process according to claim 1, wherein
R₁ is hydrogen,
R₂ is hydrogen, chlorine, —SO₃H, —SO₃⁻Mₐ⁺, carboxy, cyano, nitro or trifluoromethyl,
R₃ is hydrogen,
R₄ is hydrogen,
R₅ is hydroxy,
R₆ is hydrogen, C₁-C₅alkyl, α,α-dimethylbenzyl or a radical of formula III,
R₇ is hydrogen,
R₈ is hydrogen, C₁-C₈alkyl or —(CH₂)ₚ—COR₁₁,
R₉ is hydrogen,
R₁₁ is C₁-C₈alkoxy, 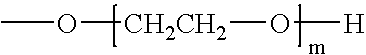
or a radical of formula IV,
R₁₅ is —O—R₁₇—O—,
R₁₇ is C₄-C₁₂alkylene interrupted by oxygen,
R₁₈ is nitro, chlorine or bromine,
M is lithium or sodium,
Mₐ⁺ is sodium or potassium,
m is an integer from 1 to 10,
n is 1, and
p is 2.

9. A process according to claim 1, wherein the reaction is carried out in a solvent.

10. A process according to claim 1, wherein the molar ratio of the amount of compound of formula V to the amount of azide compound of formula IX is from 1:1 to 1:3.

11. A process according to claim 1, wherein the reaction is carried out in the presence of a catalyst.
